(19) United States
(12) Patent Application Publication
(10) Pub. No.: US 2026/0116980 A1
(43) Pub. Date: Apr. 30, 2026

(54) ANTI-B7H3 ANTIBODIES AND METHODS OF USE

(71) Applicant: BeOne Medicines I GmbH, Basel (CH)

(72) Inventors: Xiaoyan TANG, Beijing (CN); Qi LIU, Beijing (CN); Liu XUE, Beijing (CN); Yiren XIAO, Shanghai (CN); Xiao DING, Beijing (CN); Yun CHEN, Shanghai (CN)

(73) Assignee: BeOne Medicines I GmbH, Basel (CH)

(21) Appl. No.: 19/251,439

(22) Filed: Jun. 26, 2025

Related U.S. Application Data
(63) Continuation of application No. PCT/CN2023/142813, filed on Dec. 28, 2023.

(30) Foreign Application Priority Data
Dec. 29, 2022 (WO) PCT/CN2022/143248

(51) Int. Cl. C07K 16/28; A61K 45/06; A61K 47/68; A61P 35/00

(52) U.S. Cl. CPC C07K 16/2827; A61K 45/06; A61K 47/6817; A61P 35/00; C07K 2317/31; C07K 2317/40; C07K 2317/565; C07K 2317/622; C07K 2317/732; C07K 2317/77

(57) ABSTRACT

The present disclosure provides for antibody or antigen-binding fragments thereof that bind to human 4Ig-B7H3, multispecific antibody or antigen-binding fragments thereof that recognize human 4Ig-B7H3 as one antigen and at least one other antigen, anti-human 4Ig-B7H3 antibodies or antigen-binding fragments thereof further conjugated with a cytotoxin, a pharmaceutical composition comprising said antibodies or antigen-binding fragments thereof, and use of the antibody, multispecific antibody or the composition for treating a disease, such as cancer.

### CROSS-REFERENCE TO RELATED APPLICATION

This application is a continuation of International Patent Application No. PCT/CN2023/142813, filed Dec. 28, 2023, which claims priority from International Patent Application No. PCT/CN2022/143248, filed Dec. 29, 2022, which are incorporated herein by reference in their entirety.

### SEQUENCE LISTING

The instant application contains a sequence listing which has been submitted electronically in xml format, and is hereby incorporated by reference in its entirety. Said xml file was created on Dec. 22, 2023, is 84 bytes in size, and named 138881_1340_Sequence_Listing.xml.

### FIELD OF THE DISCLOSURE

Disclosed herein are antibodies that specifically bind to human 4Ig-B7H3 as well as isolated nucleic acids, vectors and host cells. These antibodies can be used to construct multispecific antibodies with other modalities such as second tumor associated antigens (TAAs), immune checkpoints or immune stimulators, to construct antibody drug conjugates (ADCs), or to form fusion proteins. Lastly, the anti-human 4Ig-B7H3 antibodies disclosed herein can be used in the treatment of various cancers.

### BACKGROUND

B7H3 or B7-H3 (CD276 or B7RP-2) is a type I transmembrane protein identified in 2001 from a dendritic cell (DC) cDNA library (Chapoval et al., (2001) Nat. Immunol. 2 (3): 269-274). As part of the B7 immunoregulatory family, B7H3 has two isoforms in humans, 4Ig-B7H3 and 2Ig-B7H3. 4Ig-B7H3 is the main isoform expressed in malignant cells (Steinberger et al., (2004) J. Immunol. 172 (4): 2352-2359). The extracellular domain of mouse B7H3 is composed of a single pair of immunoglobulin variable (IgV)-like and immunoglobulin constant (IgC)-like domains, whereas human B7H3 contains a single pair or two identical pairs due to exon duplication (Sun et al., (2002) J. Immunol. 168 (12): 6294-6297; Steinberger et al., (2004) J. Immunol. 172 (4): 2352-2359). The intracellular domain of B7H3 is short and has no known signaling motif (Picarda et al., (2016) Clin. Cancer. Res. 22 (14): 3425-3431). Besides the transmembrane form, B7H3 may exist in a soluble form, which is cleaved from membrane B7H3 by proteinase or caused by alternative splicing (Zhang et al., (2008) Immunology. 123 (4): 538-546).

Belonging to the B7 protein family, B7H3 shares 20-27% amino-acid identity with other B7 family ligands but the receptor(s) remains to be elucidated (Chapoval et al., (2001) Nat. Immunol. 2 (3): 269-274). To date, the molecular mechanisms by which B7H3 participates in immune evasion remain elusive and B7H3 function in T cell-mediated adaptive immunity is still controversial. Although originally identified as T cell co-stimulatory molecule (Chapoval et al., (2001) Nat. Immunol. 2 (3): 269-274; Zhang et al., (2004) Acta Biochim. Biophys. Sin. 36 (6): 430-436), later studies suggest it mainly plays an immune suppressive role Suh et al., (2003) Nat. Immunol. 4 (9): 899-906; Prasad et al., (2004) J. Immunol. 173 (4): 2500-2506; Fukushima et al (2007) Immunol. Lett. 113 (1): 52-57; Veenstra et al., (2015) Blood. 125 (21): 3335-3346). Meanwhile the crystal structure of mouse B7H3 reveals that the FG Loop is important for mB7H3-mediated inhibition of T cell proliferation Vigdorovich et al., (2013) Structure. 21 (5): 707-717). Notably, NSCLC with high B7H3 expression was associated with a lower number of tumor-infiltrating lymphocytes and refractory to anti-PD1 therapy, suggesting a role for B7H3 in immune evasion. Therefore, B7H3 targeted therapy combined with anti-PD-1/PD-L1 antibody therapy is a promising approach for B7H3-expressing NSCLCs (Altan et al., (2017) Clin. Cancer. Res. 23 (17): 5202-5209).

Besides immune regulation, B7H3 also has an intrinsic protumor function. TCGA analysis reveals that B7H3 expression is correlated with the EGFR/PI3K/AKT pathway, MAPK pathway and EMT process across cancer type. Aberrant B7H3 expression promotes tumor metastasis, angiogenesis, glycolytic metabolism and drug resistance (Tekle et al., (2012) Int. J. Cancer. 130 (10): 2282-2290; Liu et al., (2015) Mol. Med. Rep. 12 (4): 5455-5460; Lee et al., (2017) Cell Res. 27 (8): 1034-1045; Flem-Karlsen et al., (2018) Trends Cancer. 4 (6): 401-404; Liu et al., (2019) Oncogene. 38 (1): 88-102; Lai et al., (2020) Immunol. Res. 68 (3): 177). Accordingly, B7H3 overexpression is correlated with poor prognosis and bad clinical outcomes in many cancer types (Crispen et al., (2008) Clin. Cancer Res. 14 (16): 5150-5157; Bachawal et al., (2015) Cancer Res. 75 (12): 2501-2509; Fan et al., (2016) Pak. J. Med. Sci. 32 (6): 1568-1573; Song et al., (2016) Onco. Targets Ther. 9:6257-6263; Wu et al., (2016) Oncotarget. 7 (49): 81750-81756; Benzon et al., (2017) Prostate Cancer Prostatic Dis. 20 (1): 28-3).

Numerous studies have reported that B7H3 is highly overexpressed in a wide range of solid tumors and tumor vasculature but has limited expression in normal tissues (Loo et al., (2012) Clin. Cancer Res. 18 (14): 3834-3845; Seaman et al., (2017) Cancer Cell. 31 (4): 501-515 e508; Du et al., (2019) Cancer Cell. 35 (2): 221-237 e228; Yamato et al., (2022) Mol. Cancer Ther. 21 (4): 635-646), making it an attractive target for cancer therapy. The majority of the B7H3 targeting assets are in early development. Y-Mabs has developed omburtamab with radionuclides including I131 or Lu177, and the lead assets is I131-omburtamab to target brain tumors. Enoblituzumab is an Fc-enhanced B7H3 Ab in phase I/II, and limited anti-tumor efficacy was demonstrated in anti-PD1/PD-L1 refractory patients when combined with an anti-PD1 antibody. B7H3 ADCs displayed preliminary anti-tumor activity in a phase I study and have a well-tolerated safety profile, including MGC018 from MacroGenics and DS-7300 from Daiichi Sankyo.

There are no approved therapeutic antibodies against B7H3, and there remains an unmet medical need for therapeutics targeting B7H3.

### SUMMARY OF THE INVENTION

The disclosure contains specific antibodies and antibody fragments thereof directed to human 4Ig-B7H3. In addition, the antibodies and antibody fragments thereof disclosed herein can be used to construct multispecific antibodies with other modalities such as a second tumor associated antigen (TAA), immune checkpoints or immune stimulators, or to construct antibody drug conjugates (ADC) or to form fusion proteins. 4Ig-B7H3 antibodies alone or in combination with other therapeutics could potentially be used for the treatment or prevention of cancer.

The present disclosure is directed to antibodies and antigen-binding fragments thereof that specifically bind human 4Ig-B7H3.

The present disclosure encompasses the following embodiments.

An antibody or antigen-binding fragment thereof which specifically binds human 4Ig-B7H3, comprising:
(i) a heavy chain variable region (VH) that comprises (a) a HCDR1 (Heavy Chain Complementarity Determining Region 1) of SEQ ID NO: 11, (b) a HCDR2 of SEQ ID NO: 2 and (c) a HCDR3 of SEQ ID NO: 14 and a light chain variable region (VL) that comprises: (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region that comprises (a) a HCDR1 of SEQ ID NO: 1, (b) a HCDR2 of SEQ ID NO: 2 and (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6;
(iii) a heavy chain variable region that comprises (a) a HCDR1 of SEQ ID NO: 11, (b) a HCDR2 of SEQ ID NO: 2 and (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6;
(iv) a heavy chain variable region that comprises (a) a HCDR1 of SEQ ID NO: 1, (b) a HCDR2 of SEQ ID NO: 2 and (c) a HCDR3 of SEQ ID NO: 14; and a light chain variable region that comprises: (d) a LCDR1 of SEQ ID NO: 4, (e) a LCDR2 of SEQ ID NO: 5, and (f) a LCDR3 of SEQ ID NO: 6;
(v) a heavy chain variable region that comprises (a) a HCDR1 of SEQ ID NO: 1, (b) a HCDR2 of SEQ ID NO: 2 and (c) a HCDR3 of SEQ ID NO: 17; and a light chain variable region that comprises: (d) a LCDR1 of SEQ ID NO: 4, (e) a LCDR2 of SEQ ID NO: 5, and (f) a LCDR3 of SEQ ID NO: 6;
(vi) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 20, (e) a LCDR2 of SEQ ID NO: 5, and (f) a LCDR3 of SEQ ID NO: 6;
(vii) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 23, (e) a LCDR2 of SEQ ID NO: 5, and (f) a LCDR3 of SEQ ID NO: 6; or
(viii) a heavy chain variable region that comprises (a) a HCDR1 of SEQ ID NO: 11, (b) a HCDR2 of SEQ ID NO: 2 and (c) a HCDR3 of SEQ ID NO: 28; and a light chain variable region that comprises: (d) a LCDR1 of SEQ ID NO: 23, (e) a LCDR2 of SEQ ID NO: 5, and (f) a LCDR3 of SEQ ID NO: 6.

The antibody or antigen-binding fragment thereof, wherein:
(i) the heavy chain variable region comprises an amino acid sequence at least 90, 91, 92, 93, 94, 95, 96, 97, 98 or 99% identical to SEQ ID NO: 26, and the light chain variable region comprises an amino acid sequence at least 90, 91, 92, 93, 94, 95, 96, 97, 98 or 99% identical to SEQ ID NO: 24;
(ii) the heavy chain variable region comprises an amino acid sequence at least 90, 91, 92, 93, 94, 95, 96, 97, 98 or 99% identical to SEQ ID NO: 7, and the light chain variable region comprises an amino acid sequence at least 90, 91, 92, 93, 94, 95, 96, 97, 98 or 99% identical to SEQ ID NO: 8;
(iii) the heavy chain variable region comprises an amino acid sequence at least 90, 91, 92, 93, 94, 95, 96, 97, 98 or 99% identical to SEQ ID NO: 12, and the light chain variable region comprises an amino acid sequence at least 90, 91, 92, 93, 94, 95, 96, 97, 98 or 99% identical to SEQ ID NO: 8;
(iv) the heavy chain variable region comprises an amino acid sequence at least 90, 91, 92, 93, 94, 95, 96, 97, 98 or 99% identical to SEQ ID NO: 15, and the light chain variable region comprises an amino acid sequence at least 90, 91, 92, 93, 94, 95, 96, 97, 98 or 99% identical to SEQ ID NO: 8;
(v) the heavy chain variable region comprises an amino acid sequence at least 90, 91, 92, 93, 94, 95, 96, 97, 98 or 99% identical to SEQ ID NO: 18, and the light chain variable region comprises an amino acid sequence at least 90, 91, 92, 93, 94, 95, 96, 97, 98 or 99% identical to SEQ ID NO: 8;
(vi) the heavy chain variable region comprises an amino acid sequence at least 90, 91, 92, 93, 94, 95, 96, 97, 98 or 99% identical to SEQ ID NO: 7, and the light chain variable region comprises an amino acid sequence at least 90, 91, 92, 93, 94, 95, 96, 97, 98 or 99% identical to SEQ ID NO: 21;
(vii) the heavy chain variable region comprises an amino acid sequence at least 90, 91, 92, 93, 94, 95, 96, 97, 98 or 99% identical to SEQ ID NO: 7, and the light chain variable region comprises an amino acid sequence at least 90, 91, 92, 93, 94, 95, 96, 97, 98 or 99% identical to SEQ ID NO: 24; or
(viii) the heavy chain variable region comprises an amino acid sequence at least 90, 91, 92, 93, 94, 95, 96, 97, 98 or 99% identical to SEQ ID NO: 29, and the light chain variable region comprises an amino acid sequence at least 90, 91, 92, 93, 94, 95, 96, 97, 98 or 99% identical to SEQ ID NO: 24.

The antibody or antigen-binding fragment thereof, wherein one, two, three, four, five, six, seven, eight, nine, or ten amino acids within SEQ ID NOs: 26 and 24, SEQ ID NOs: 7 and 8, SEQ ID NOs: 12 and 8, SEQ ID NOs: 15 and 8, SEQ ID NOs: 18 and 8, SEQ ID NOs: 7 and 21, SEQ ID Nos: 7 and 24, SEQ ID Nos: 29 and 24 have been inserted, deleted or substituted.

The antibody or antigen-binding fragment thereof, wherein:
(i) the heavy chain variable region comprises SEQ ID NO: 26, and the light chain variable region comprises SEQ ID NO: 24;
(ii) the heavy chain variable region comprises SEQ ID NO: 7, and the light chain variable region comprises SEQ ID NO: 8;
(iii) the heavy chain variable region comprises SEQ ID NO: 12, and the light chain variable region comprises SEQ ID NO: 8;
(iv) the heavy chain variable region comprises SEQ ID NO: 15, and the light chain variable region comprises SEQ ID NO: 8;
(v) the heavy chain variable region comprises SEQ ID NO: 18, and the light chain variable region comprises SEQ ID NO: 8;
(vi) the heavy chain variable region comprises SEQ ID NO: 7, and the light chain variable region comprises SEQ ID NO: 21;
(vii) the heavy chain variable region comprises SEQ ID NO: 7, and the light chain variable region comprises SEQ ID NO: 24; or
(viii) the heavy chain variable region comprises SEQ ID NO: 29, and the light chain variable region comprises SEQ ID NO: 24.

The antibody or antigen-binding fragment thereof, which is a monoclonal antibody, a human engineered antibody, a single chain antibody (scFv), a Fab fragment, a Fab′ fragment, or a F(ab′)2 fragment.

The antibody or antigen-binding fragment thereof, wherein the antibody or antigen-binding fragment thereof comprises a scFv comprising a VH having the amino acid of SEQ ID NO: 26 and a VL having an amino acid of SEQ ID NO: 24, optionally the VH and VL are connected via an amino acid linker, optionally the amino acid linker is any sequence of SEQ ID NO: 35 to SEQ ID NO: 77.

The antibody or antigen-binding fragment thereof, wherein the amino acid linker is SEQ ID NO: 77.

The antibody or antigen-binding fragment thereof, wherein the antibody or antigen-binding fragment thereof comprises a scFv having the amino acid sequence of SEQ ID NO: 32.

A multispecific antibody or antigen-binding fragment thereof comprising at least a first antigen binding domain that specifically binds human 4Ig-B7H3, wherein the first antigen binding domain are the antibody or antigen-binding fragment thereof described herein; and at least a second antigen binding domain that specifically binds a second human tumor-associated antigen (TAA).

The multispecific antibody or antigen-binding fragment thereof, wherein the multispecific antibody is a bispecific antibody.

The multispecific antibody or antigen-binding fragment thereof, further comprising an amino acid linker, wherein the amino acid linker is any sequence of SEQ ID NO: 35 to SEQ ID NO: 77.

The antibody or antigen-binding fragment thereof, wherein the antibody or antigen-binding fragment thereof comprises a heavy chain constant region of the subclass of IgG1, IgG2, IgG3, or IgG4, and/or a light chain constant region of the type of kappa or lambda.

The antibody or antigen-binding fragment thereof, wherein the antibody or antigen-binding fragment thereof comprises a heavy chain constant region of the subclass of IgG1, and a light chain constant region of the type of kappa.

The antibody or antigen-binding fragment thereof, wherein the antibody or antigen-binding fragment thereof has antibody dependent cellular cytotoxicity (ADCC) or complement dependent cytotoxicity (CDC).

The antibody or antigen-binding fragment thereof, wherein the antibody or antigen-binding fragment thereof has reduced glycosylation or no glycosylation or is hypofucosylated.

The antibody or antigen-binding fragment thereof, wherein the antibody or antigen-binding fragment thereof comprises increased bisecting GlcNac structures.

The antibody or antigen-binding fragment thereof, wherein the antibody or antigen-binding fragment thereof is conjugated to a cytotoxin.

The antibody or antigen-binding fragment thereof, wherein the antibody or antigen-binding fragment thereof is conjugated to a cytotoxin via a cytotoxin linker.

The antibody or antigen-binding fragment thereof, wherein:
(1) the antibody or antigen-binding fragment thereof specifically binds to an epitope comprising or consisting of amino acid residues 29-139 of human 4Ig-B7H3 (SEQ ID NO: 80); and/or
(2) the antibody or antigen-binding fragment thereof specifically binds to an epitope comprising or consisting of amino acid residues 243-357 of human 4Ig-B7H3 (SEQ ID NO: 80).

In some embodiments, (3) the antibody or antigen-binding fragment thereof does not bind to an epitope comprising or consisting of amino acid residues 145-238 of human 4Ig-B7H3 (SEQ ID NO: 80); and/or (4) the antibody or antigen-binding fragment thereof does not bind to an epitope comprising or consisting of amino acid residues 363-456 of human 4Ig-B7H3 (SEQ ID NO: 80).

A pharmaceutical composition comprising the antibody or antigen-binding fragment thereof described herein and a pharmaceutically acceptable carrier.

A method of treating a cancer comprising administering to a patient in need thereof an effective amount of the antibody or antigen-binding fragment thereof, or the pharmaceutical composition described herein.

The method, wherein the cancer is 4Ig-B7H3 positive.

The method, wherein the cancer is colorectal carcinoma, prostate cancer, pancreatic cancer, breast cancer, ovarian cancer, kidney cancer, lung cancer, or esophageal carcinoma.

The method, wherein the lung cancer is non-small cell lung cancer (NSCLC) or small cell lung cancer (SCLC).

The method, wherein the non-small cell lung cancer is squamous non-small cell lung cancer.

The method, wherein the esophageal carcinoma is esophageal squamous cell carcinoma.

The method, wherein the antibody or antigen-binding fragment thereof is administered in combination with another therapeutic agent.

The method, wherein the therapeutic agent is paclitaxel or a paclitaxel agent, docetaxel, carboplatin, topotecan, cisplatin, irinotecan, doxorubicin, lenalidomide or 5-azacytidine. The method, wherein the therapeutic agent is an immune checkpoint inhibitor.

The method, wherein the therapeutic agent is an anti-PD-1 antibody.

The method, wherein the anti-PD1 antibody is Tislelizumab.

An isolated nucleic acid that encodes the antibody or antigen-binding fragment thereof described herein.

A vector comprising the nucleic acid described herein.

A host cell comprising the nucleic acid or the vector described herein.

A process for producing an antibody or antigen-binding fragment thereof comprising cultivating the host cell described herein and recovering the antibody or antibody fragment from the culture.

An antibody or antigen-binding fragment thereof which specifically binds human 4Ig-B7H3, wherein:
(1) the antibody or antigen-binding fragment thereof specifically binds to an epitope comprising or consisting of amino acid residues 29-139 of human 4Ig-B7H3 (SEQ ID NO: 80); and/or
(2) the antibody or antigen-binding fragment thereof specifically binds to an epitope comprising or consisting of amino acid residues 243-357 of human 4Ig-B7H3 (SEQ ID NO: 80).

In some embodiments, (3) the antibody or antigen-binding fragment thereof does not bind to an epitope comprising or consisting of amino acid residues 145-238 of human 4Ig-B7H3 (SEQ ID NO: 80); and/or (4) the antibody or antigen-binding fragment thereof does not bind to an epitope comprising or consisting of amino acid residues 363-456 of human 4Ig-B7H3 (SEQ ID NO: 80).

In some embodiments, the present disclosure provides anti-human 4Ig-B7H3 antibodies or antigen-binding fragments thereof which show specific binding and high affinity to human 4Ig-B7H3.

In some embodiments, the present disclosure provides anti-human 4Ig-B7H3 antibodies or antigen-binding fragments thereof which show increased binding affinity and/or increased internalization (such as an increased internalization rate).

### BRIEF DESCRIPTION OF THE DRAWINGS

FIG. 1 shows the affinity determination of purified B7H3 mAb BGA-3726 (murine chimeric) on human B7H3 antigen by SPR. Specifically, the sensorgram shows the changes in binding as a signal originating from the surface of a glass chip. The glass chip is a plasmon resonance surface, bound with BGA-3726. Anti-BGA-3726 antibodies were captured by anti-mouse IgG Fc antibody and immobilized on the chip. After immobilization, serial dilutions of the ectodomain of human or cyno B7H3 were passed over the chip. As B7H3 is the target of BGA-3726, the degree of binding is visualized as Response Units (RU) (Y-axis) and is proportional to binding over time in seconds (X-axis). Thus, the light gray lines are serial dilutions of human B7H3 protein, and the dark gray lines are fitted curves corresponding to different human B7H3 concentrations. The dotted line is the highest concentration of B7H3 protein. The results were used to calculate the rates of association (Kon) and dissociation (Koff) for BGA-3295.

FIGS. 2A-B show the binding affinity of purified anti-4Ig-B7H3 mAb BGA-3726 with human 4Ig-B7H3 (FIG. 2A) and cyno B7H3 (FIG. 2B) overexpressing cells by FACS assay.

FIGS. 3A-B show the binding affinities comparison of engineered antibodies derived from BGA-3726 to human 4Ig-B7H3 overexpressing cells by FACS assay. FIG. 3A shows the dose dependent binding curves of BGA-3726 to human 4Ig-B7H3 overexpressing cells. FIG. 3B shows the dose dependent binding curves of BGA-6938 and BGA-5488 to human 4Ig-B7H3 overexpressing cells.

FIG. 4 shows the binding affinities comparison of BGA-6938 and MABX-9001a to B7H3 positive tumor cells H358 cells by FACS assay. hIgG isotype Ab was used as non-binding control.

FIG. 5 shows the internalization activity comparison of BGA-6938 and MABX-9001a to B7H3 positive tumor cells H358 cells by FACS assay at indicated time point. hIgG isotype Ab was used as non-binding control.

FIG. 6 shows the cell killing activity of BGA-6938-GGFG-DXd (DAR8), MABX-9001a-GGFG-DXd (DAR8) and MABX-9001a-GGFG-DXd (DAR4) in H1650, H441 and H1048 cell models. Iso-GGFG-DXd (DAR8) was used as non-targeting control.

FIG. 7 shows the binding affinity of scFv of BGA-6938 to H358 cells.

FIGS. 8A-B shows epitope mapping of BGB-6938. FIG. 8A shows the construct design of truncated human B7H3. FIG. 8B shows the epitope identification of BGB-6938 by domain truncation and comparison of the epitope with DS-7300.

### DETAILED DESCRIPTION

The present disclosure provides for anti-human 4Ig-B7H3 antibodies and antigen-binding fragments thereof. Also, the present disclosure provides antibodies that have desirable binding affinity, desirable internalization (such as an increased internalization rate) and other desirable attributes. The anti-human 4Ig-B7H3 antibodies can be used to construct multispecific antibodies with other modalities such as second tumor associated antigens (TAAs), immune checkpoints or immune stimulators, or to construct antibody drug conjugates (ADCs), or to fuse with other domains to form fusion proteins. Furthermore, the anti-human 4Ig-B7H3 antibodies and the constructs thereof could be used for treating cancer and associated disorders.

### I. Anti-4Ig-B7H3 Antibodies

The present disclosure provides for antibodies or antigen-binding fragments thereof that specifically bind to human 4Ig-B7H3. Antibodies or antigen-binding fragments of the present disclosure include, but are not limited to, the antibodies or antigen-binding fragments thereof, generated as described below.

The present disclosure provides for antibodies or antigen-binding fragments that specifically bind to human 4Ig-B7H3, wherein said antibodies or antibody fragments (e.g., antigen-binding fragments) comprise a VH domain having an amino acid sequence of SEQ ID NO: 26, SEQ ID NO: 7, SEQ ID NO: 12, SEQ ID NO: 15, SEQ ID NO: 18, or SEQ ID NO: 29 (Table 7). The present disclosure also provides antibodies or antigen-binding fragments that specifically bind human 4Ig-B7H3, wherein said antibodies or antigen-binding fragments comprise a HCDR having an amino acid sequence of any one of the HCDRs listed in Table 7. In one aspect, the present disclosure provides antibodies or antigen-binding fragments that specifically bind to human 4Ig-B7H3, wherein said antibodies comprise one, two, three, or more HCDRs having an amino acid sequence of any of the HCDRs listed in Table 7.

The present disclosure provides for antibodies or antigen-binding fragments that specifically bind to human 4Ig-B7H3, wherein said antibodies or antibody fragments (e.g., antigen-binding fragments) comprise a VL domain having an amino acid sequence of SEQ ID NO: 24, SEQ ID NO: 8, or SEQ ID NO: 21 (Table 7). The present disclosure also provides antibodies or antigen-binding fragments that specifically bind human 4Ig-B7H3, wherein said antibodies or antigen-binding fragments comprise a LCDR having an amino acid sequence of any one of the LCDRs listed in Table 7. In one aspect, the present disclosure provides antibodies or antigen-binding fragments that specifically bind to human 4Ig-B7H3, wherein said antibodies comprise one, two, three, or more LCDRs having an amino acid sequence of any of the LCDRs listed in Table 7.

In one embodiment, the antibody or an antigen-binding fragment thereof that specifically bind to human 4Ig-B7H3 comprises one or more complementarity determining regions (CDRs) comprising an amino acid sequence selected from a group consisting of SEQ ID NO: 1, SEQ ID NO: 2, SEQ ID NO: 3, SEQ ID NO: 11, SEQ ID NO: 14; SEQ ID NO: 17, SEQ ID NO: 28, SEQ ID NO: 4, SEQ ID NO: 5, SEQ ID NO: 6, SEQ ID NO: 20, and SEQ ID NO: 23.

In another embodiment, the antibody or an antigen-binding fragment thereof that specifically bind to human 4Ig-B7H3 comprises: (a) a heavy chain variable region comprising one or more complementarity determining regions (HCDRs) comprising an amino acid sequence selected from the group consisting of SEQ ID NO: 1, SEQ ID NO: 2, SEQ ID NO: 3, SEQ ID NO: 11, SEQ ID NO: 14; SEQ ID NO: 17, and SEQ ID NO: 28, and/or (b) a light chain variable region comprising one or more complementarity determining regions (LCDRs) comprising an amino acid sequence selected from the group consisting of SEQ ID NO: 4, SEQ ID NO: 5, SEQ ID NO: 6, SEQ ID NO: 20, and SEQ ID NO: 23.

In another embodiment, the antibody or an antigen-binding fragment thereof that specifically bind to human 4Ig-B7H3 comprises: (a) a heavy chain variable region comprising three complementarity determining regions (HCDRs) which are HCDR1 comprising an amino acid sequence of SEQ ID NO: 1 or SEQ ID NO: 11; HCDR2 comprising an amino acid sequence of SEQ ID NO: 2; and HCDR3 comprising an amino acid sequence of SEQ ID NO: 3, SEQ ID NO: 14, SEQ ID NO: 28, or SEQ ID NO: 17; and/or (b) a light chain variable region comprising three complementarity determining regions (LCDRs) which are LCDR1 comprising an amino acid sequence of SEQ ID NO: 4, SEQ ID NO: 20, or SEQ ID NO: 23; LCDR2 comprising an amino acid sequence of SEQ ID NO: 5; and LCDR3 comprising an amino acid sequence of SEQ ID NO: 6.

In another embodiment, the antibody or an antigen-binding fragment thereof that specifically bind to human 4Ig-B7H3 comprises: (a) a heavy chain variable region comprising three complementarity determining regions (HCDRs) which are: HCDR1 comprising an amino acid sequence of SEQ ID NO: 1, HCDR2 comprising an amino acid sequence of SEQ ID NO: 2, and HCDR3 comprising an amino acid sequence of SEQ ID NO: 3; HCDR1 comprising an amino acid sequence of SEQ ID NO: 11, HCDR2 comprising an amino acid sequence of SEQ ID NO: 2, and HCDR3 comprising an amino acid sequence of SEQ ID NO: 3; HCDR1 comprising an amino acid sequence of SEQ ID NO: SEQ ID NO: 1, HCDR2 comprising an amino acid sequence of SEQ ID NO: 2, and HCDR3 comprising an amino acid sequence of SEQ ID NO: 14; HCDR1 comprising an amino acid sequence of SEQ ID NO: SEQ ID NO: 1, HCDR2 comprising an amino acid sequence of SEQ ID NO: 2, and HCDR3 comprising an amino acid sequence of SEQ ID NO: 17; HCDR1 comprising an amino acid sequence of SEQ ID NO: 11, HCDR2 comprising an amino acid sequence of SEQ ID NO: 2, and HCDR3 comprising an amino acid sequence of SEQ ID NO: 14; or HCDR1 comprising an amino acid sequence of SEQ ID NO: SEQ ID NO: 11, HCDR2 comprising an amino acid sequence of SEQ ID NO: 2, and HCDR3 comprising an amino acid sequence of SEQ ID NO: 28 and/or (b) a light chain variable region comprising three complementarity determining regions (LCDRs) which are LCDR1 comprising an amino acid sequence of SEQ ID NO: 4, LCDR2 comprising an amino acid sequence of SEQ ID NO: 5, and LCDR3 comprising an amino acid sequence of SEQ ID NO: 6; LCDR1 comprising an amino acid sequence of SEQ ID NO: 20, LCDR2 comprising an amino acid sequence of SEQ ID NO: 5, and LCDR3 comprising an amino acid sequence of SEQ ID NO: 6; or LCDR1 comprising an amino acid sequence of SEQ ID NO: 23, LCDR2 comprising an amino acid sequence of SEQ ID NO: 5, and LCDR3 comprising an amino acid sequence of SEQ ID NO: 6.

In one embodiment, the antibodies or antigen-binding fragments thereof that specifically bind to human 4Ig-B7H3 comprise: (i) a HCDR1 (Heavy Chain Complementarity Determining Region 1), a HCDR2 and a HCDR3 from the heavy chain variable region (VH) set forth in SEQ ID NO: 26; (ii) a HCDR1, a HCDR2 and a HCDR3 from the heavy chain variable region (VH) set forth in SEQ ID NO: 7; (iii) a HCDR1, a HCDR2 and a HCDR3 from the heavy chain variable region (VH) set forth in SEQ ID NO: 12; (iv) a HCDR1, a HCDR2 and a HCDR3 from the heavy chain variable region (VH) set forth in SEQ ID NO: 15; (v). a HCDR1, a HCDR2 and a HCDR3 from the heavy chain variable region (VH) set forth in SEQ ID NO: 18; or (vi) a HCDR1, a HCDR2 and a HCDR3 from the heavy chain variable region (VH) set forth in SEQ ID NO: 29; and/or (i) a LCDR1 (Light Chain Complementarity Determining Region 1), a LCDR2 and a LCDR3 from the light chain variable region (VL) set forth in SEQ ID NO: 24; (ii) a LCDR1, a LCDR2 and a LCDR3 from the light chain variable region (VL) set forth in SEQ ID NO: 21; (iii) a LCDR1, a LCDR2 and a LCDR3 from the light chain variable region (VL) set forth in SEQ ID NO: 8.

In one embodiment, the antibodies or antigen-binding fragments thereof that specifically bind to human 4Ig-B7H3 comprise:
(i) a heavy chain variable region (VH) that comprises (a) a HCDR1 (Heavy Chain Complementarity Determining Region 1) of SEQ ID NO: 11, (b) a HCDR2 of SEQ ID NO: 2 and (c) a HCDR3 of SEQ ID NO: 14 and a light chain variable region (VL) that comprises: (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region that comprises (a) a HCDR1 of SEQ ID NO: 1, (b) a HCDR2 of SEQ ID NO: 2 and (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6;
(iii) a heavy chain variable region that comprises (a) a HCDR1 of SEQ ID NO: 11, (b) a HCDR2 of SEQ ID NO: 2 and (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6;
(iv) a heavy chain variable region that comprises (a) a HCDR1 of SEQ ID NO: 1, (b) a HCDR2 of SEQ ID NO: 2 and (c) a HCDR3 of SEQ ID NO: 14; and a light chain variable region that comprises: (d) a LCDR1 of SEQ ID NO: 4, (e) a LCDR2 of SEQ ID NO: 5, and (f) a LCDR3 of SEQ ID NO: 6;
(v) a heavy chain variable region that comprises (a) a HCDR1 of SEQ ID NO: 1, (b) a HCDR2 of SEQ ID NO: 2 and (c) a HCDR3 of SEQ ID NO: 17; and a light chain variable region that comprises: (d) a LCDR1 of SEQ ID NO: 4, (e) a LCDR2 of SEQ ID NO: 5, and (f) a LCDR3 of SEQ ID NO: 6;
(vi) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 20, (e) a LCDR2 of SEQ ID NO: 5, and (f) a LCDR3 of SEQ ID NO: 6;
(vii) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 23, (e) a LCDR2 of SEQ ID NO: 5, and (f) a LCDR3 of SEQ ID NO: 6; or
(viii) a heavy chain variable region that comprises (a) a HCDR1 of SEQ ID NO: 11, (b) a HCDR2 of SEQ ID NO: 2 and (c) a HCDR3 of SEQ ID NO: 28; and a light chain variable region that comprises: (d) a LCDR1 of SEQ ID NO: 23, (e) a LCDR2 of SEQ ID NO: 5, and (f) a LCDR3 of SEQ ID NO: 6, according to the Kabat definition.

In one embodiment, the antibody or an antigen-binding fragment thereof of the present disclosure comprises: (a) a heavy chain variable region comprising an amino acid sequence of SEQ ID NO: 26, SEQ ID NO: 7, SEQ ID NO: 12, SEQ ID NO: 15, SEQ ID NO: 18, or SEQ ID NO: 29 or an amino acid sequence at least 90%, 91%, 92%, 93%, 94%, 95%, 96%, 97%, 98% or 99% identical to any one of SEQ ID NO: 26, SEQ ID NO: 7, SEQ ID NO: 12, SEQ ID NO: 15, SEQ ID NO: 18, or SEQ ID NO: 29 and/or (b) a light chain variable region comprising an amino acid sequence of SEQ ID NO: 24, SEQ ID NO: 8, or SEQ ID NO: 21, or an amino acid sequence at least 90%, 91%, 92%, 93%, 94%, 95%, 96%, 97%, 98% or 99% identical to any one of SEQ ID NO: 24, SEQ ID NO: 8, or SEQ ID NO: 21.

In one embodiment, the antibody or antigen-binding fragment thereof comprises:
(i) a heavy chain variable region comprising an amino acid sequence at least 90, 91, 92, 93, 94, 95, 96, 97, 98 or 99% identical to SEQ ID NO: 26, and a light chain variable region comprising an amino acid sequence at least 90, 91, 92, 93, 94, 95, 96, 97, 98 or 99% identical to SEQ ID NO: 24;
(ii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 8;
(iii) a heavy chain variable region comprising an amino acid sequence at least 90, 91, 92, 93, 94, 95, 96, 97, 98 or 99% identical to SEQ ID NO: 12, and a light chain variable region comprising an amino acid sequence at least 90, 91, 92, 93, 94, 95, 96, 97, 98 or 99% identical to SEQ ID NO: 8;
(iv) a heavy chain variable region comprising an amino acid sequence at least 90, 91, 92, 93, 94, 95, 96, 97, 98 or 99% identical to SEQ ID NO: 15, and a light chain variable region comprising an amino acid sequence at least 90, 91, 92, 93, 94, 95, 96, 97, 98 or 99% identical to SEQ ID NO: 8;
(v) a heavy chain variable region comprising an amino acid sequence at least 90, 91, 92, 93, 94, 95, 96, 97, 98 or 99% identical to SEQ ID NO: 18, and a light chain variable region comprising an amino acid sequence at least 90, 91, 92, 93, 94, 95, 96, 97, 98 or 99% identical to SEQ ID NO: 8;
(vi) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 21;
(vii) a heavy chain variable region comprising an amino acid sequence at least 90, 91, 92, 93, 94, 95, 96, 97, 98 or 99% identical to SEQ ID NO: 7, and a light chain variable region comprising an amino acid sequence at least 90, 91, 92, 93, 94, 95, 96, 97, 98 or 99% identical to SEQ ID NO: 24; or
(viii) a heavy chain variable region comprising an amino acid sequence at least 90, 91, 92, 93, 94, 95, 96, 97, 98 or 99% identical to SEQ ID NO: 29, and a light chain variable region comprising an amino acid sequence at least 90, 91, 92, 93, 94, 95, 96, 97, 98 or 99% identical to SEQ ID NO: 24.

In one embodiment, the present disclosure provides an antibody or antigen-binding fragment thereof, wherein one, two, three, four, five, six, seven, eight, nine, or ten amino acids within SEQ ID NOs: 26 and 24, SEQ ID NOs: 7 and 8, SEQ ID NOs: 12 and 8, SEQ ID NOs: 15 and 8, SEQ ID NOs: 18 and 8, SEQ ID NOs: 7 and 21, SEQ ID NOs: 7 and 24, SEQ ID NOs: 29 and 24 have been inserted, deleted or substituted.

In one embodiment, the antibody or antigen-binding fragment thereof, that comprises:
(i) a heavy chain variable region comprising SEQ ID NO: 26, and a light chain variable region comprising SEQ ID NO: 24;
(ii) a heavy chain variable region comprising SEQ ID NO: 7, and a light chain variable region comprising SEQ ID NO: 8;
(iii) a heavy chain variable region comprising SEQ ID NO: 12, and a light chain variable region comprising SEQ ID NO: 8;
(iv) a heavy chain variable region comprising SEQ ID NO: 15, and a light chain variable region comprising SEQ ID NO: 8;
(v) a heavy chain variable region comprising SEQ ID NO: 18, and a light chain variable region comprising SEQ ID NO: 8;
(vi) a heavy chain variable region comprising SEQ ID NO: 7, and a light chain variable region comprising SEQ ID NO: 21;
(vii) a heavy chain variable region comprising SEQ ID NO: 7, and a light chain variable region comprising SEQ ID NO: 24; or
(viii) a heavy chain variable region comprising SEQ ID NO: 29, and a light chain variable region comprising SEQ ID NO: 24.

Other antibodies or antigen-binding fragments thereof of the present disclosure include amino acids that have been changed, yet have at least 60%, 70%, 80%, 90%, 91%, 92%, 93%, 94%, 95% 96%, 97%, 98%, or 99% percent identity in the CDR regions compared with the CDR regions disclosed in Table 7. In some aspects, it includes amino acid changes (insertion, deletion or substitution, optionally conservative amino acid substitutions) wherein no more than 1, 2, 3, 4 or 5 amino acids have been changed in the CDR regions when compared with the CDR regions depicted in the sequence described in Table 7, while maintaining binding specificity and affinity.

Other antibodies of the present disclosure include those where the amino acids or nucleic acids encoding the amino acids have been changed in the variable regions (e.g., the frameworks regions of the variable regions); yet have at least 60, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% percent identity to the sequences of variable regions described in Table 7, while retaining binding specificity/affinity, optionally the corresponding sequences of CDRs do not change. In some aspects, it includes changes in the amino acid sequences wherein no more than 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids have been changed in the variable regions (e.g., the frameworks regions of the variable regions) when compared with the variable regions depicted in the sequence described in Table 7, while retaining binding specificity/affinity, optionally the corresponding sequences of CDRs do not change.

In another embodiment, the present disclosure provides for antibodies or antigen-binding fragments thereof that specifically bind to human 4Ig-B7H3 with a binding affinity (K_{D}) of from 1×10⁻⁶ M to 1×10⁻¹¹ M. In another embodiment, the anti-4Ig-B7H3 antibodies or antigen-binding fragments thereof bind to human 4Ig-B7H3 with a binding affinity (K_{D}) of about 1×10⁻⁶ M, about 1×10⁻⁷ M, about 1×10⁻⁸ M, about 1×10⁻⁹ M, about 1×10⁻¹⁰ M or about 1×10⁻¹¹ M.

The present disclosure also provides nucleic acid sequences that encode VH or VL of the antibodies that specifically bind to human 4Ig-B7H3. Such nucleic acid sequences can be optimized for expression in mammalian cells.

The present disclosure also provides for antibodies and antigen-binding fragments thereof that bind to the same epitope as do the anti-4Ig-B7H3 antibodies described in Table 7. Additional antibodies and antigen-binding fragments thereof can therefore be identified based on their ability to cross-compete (e.g., to competitively inhibit the binding of, in a statistically significant manner) with the antibodies described in Table 7 in binding assays. The ability of a test antibody to inhibit the binding of antibodies and antigen-binding fragments thereof of the present disclosure to 4Ig-B7H3 demonstrates that the test antibody can compete with that antibody or antigen-binding fragments thereof for binding to 4Ig-B7H3. Such an antibody can, without being bound to any one theory, bind to the same or a related (e.g., a structurally similar or spatially proximal) epitope on 4Ig-B7H3 as the antibody or antigen-binding fragments thereof with which it competes. In a certain aspect, the antibody that binds to the same epitope on 4Ig-B7H3 as the antibodies or antigen-binding fragments thereof of the present disclosure is a human or humanized monoclonal antibody. Such human or humanized monoclonal antibodies can be prepared and isolated as described herein.

In some embodiments, the present disclosure provides for an antibody or antigen-binding fragment thereof that specifically bind to human 4Ig-B7H3, wherein the antibody or antigen-binding fragment thereof specifically binds to an epitope comprising, consisting essentially of or consisting of amino acid residues 29-139 of human 4Ig-B7H3 (SEQ ID NO: 80).

In some embodiments, the present disclosure provides for an antibody or antigen-binding fragment thereof that specifically bind to human 4Ig-B7H3, wherein the antibody or antigen-binding fragment thereof specifically binds to an epitope comprising, consisting essentially of or consisting of amino acid residues 243-357 of human 4Ig-B7H3 (SEQ ID NO: 80).

In some embodiments, the present disclosure provides for an antibody or antigen-binding fragment thereof that specifically bind to human 4Ig-B7H3, wherein the antibody or antigen-binding fragment thereof specifically binds to IgV1 domain of human 4Ig-B7H3. In some embodiments, the present disclosure provides for an antibody or antigen-binding fragment thereof that specifically bind to human 4Ig-B7H3, wherein the antibody or antigen-binding fragment thereof specifically binds to IgV2 domain of human 4Ig-B7H3. In some embodiments, the present disclosure provides for an antibody or antigen-binding fragment thereof that specifically bind to human 4Ig-B7H3, wherein the antibody or antigen-binding fragment thereof binds to both IgV1 and IgV2 domains of human 4Ig-B7H3.

In some embodiments, the present disclosure provides for an antibody or antigen-binding fragment thereof that specifically bind to human 4Ig-B7H3, wherein the antibody or antigen-binding fragment thereof does not bind to an epitope comprising, consisting essentially of or consisting of amino acid residues 145-238 of human 4Ig-B7H3 (SEQ ID NO: 80).

In some embodiments, the present disclosure provides for an antibody or antigen-binding fragment thereof that specifically bind to human 4Ig-B7H3, wherein the antibody or antigen-binding fragment thereof does not bind to an epitope comprising, consisting essentially of or consisting of amino acid residues 363-456 of human 4Ig-B7H3 (SEQ ID NO: 80).

In some embodiments, the present disclosure provides for an antibody or antigen-binding fragment thereof that specifically bind to human 4Ig-B7H3, wherein the antibody or antigen-binding fragment thereof does not bind to IgC1 domain of human 4Ig-B7H3. In some embodiments, the present disclosure provides for an antibody or antigen-binding fragment thereof that specifically bind to human 4Ig-B7H3, wherein the antibody or antigen-binding fragment thereof does not bind to IgC2 domain of human 4Ig-B7H3. In some embodiments, the present disclosure provides for an antibody or antigen-binding fragment thereof that specifically bind to human 4Ig-B7H3, wherein the antibody or antigen-binding fragment thereof binds to neither IgC1 nor IgC2 domain of human 4Ig-B7H3.

In some embodiments, the present disclosure provides for an antibody or antigen-binding fragment thereof that specifically bind to human 4Ig-B7H3, wherein the antibody or antigen-binding fragment thereof specifically binds to an epitope that does not overlap with the epitope of reference antibody DS-7300.

In some embodiments, the antibody or antigen-binding fragment thereof is a monoclonal antibody, a human engineered antibody, a single chain antibody (scFv), a Fab fragment, a Fab′ fragment, or a F(ab′)2 fragment.

In one embodiment, the antibody or antigen-binding fragment thereof is in a scFv format comprising VH-VL in the direction of N-terminal to C-terminal, or VL-VH in the direction of N-terminal to C-terminal. In some embodiments, the VH and VL are connected via an amino acid linker described herein. In some embodiments, the VH comprises the amino acid sequence of any one of SEQ ID NO: 26, SEQ ID NO: 7, SEQ ID NO: 12, SEQ ID NO: 15, SEQ ID NO: 18, or SEQ ID NO: 29. In some embodiments, the VL comprises the amino acid sequence of any one of SEQ ID NO: 24, SEQ ID NO: 8, or SEQ ID NO: 21. In some embodiments, the VH is any one of VHs described in Table 7. In some embodiment, the VL is any one of VLs described in Table 7. In some embodiments, the amino acid linker has an amino acid sequence comprising any one of SEQ ID NO: 35-77. In some embodiments, the amino acid linker is any sequence of SEQ ID NO: 35-77. In one embodiment, the amino acid linker is SEQ ID NO: 77.

In one embodiment, the antibody or antigen-binding fragment thereof comprises a scFv having the amino acid sequence of SEQ ID NO: 32. In another embodiment, the antibody or antigen-binding fragment thereof comprises a scFv of SEQ ID NO: 32.

### II. Anti-4Ig-B7H3 Multispecific Antibodies

In one embodiment, the anti-4Ig-B7H3 antibodies as disclosed herein can be used to construct multispecific antibodies with other modalities such as a second human tumor associated antigen (TAA), immune checkpoints or immune stimulators, wherein 4Ig-B7H3 works as a first TAA.

In one embodiment, the anti-4Ig-B7H3 antibodies as disclosed herein can be incorporated into an anti-4Ig-B7H3xTAA multispecific antibody, wherein TAA is an antibody or fragment thereof directed to any human tumor associated antigen. An antibody molecule is a multispecific antibody molecule, for example, it comprises a number of antigen binding domains, wherein at least one antigen binding domain sequence specifically binds 4Ig-B7H3 as a first epitope and a second antigen binding domain sequence specifically binds a second TAA as a second epitope. In one embodiment, the multispecific antibody comprises a third, fourth or fifth antigen binding domain. In one embodiment, the multispecific antibody is a bispecific antibody, a trispecific antibody, or tetraspecific antibody.

In one embodiment, the multispecific antibody is a bispecific antibody. As used herein, a bispecific antibody specifically binds only two antigens. The bispecific antibody comprises a first antigen binding domain which specifically binds human 4Ig-B7H3 and a second antigen binding domain that specifically binds a second TAA. This includes a bispecific antibody comprising a second heavy chain variable domain and a second light chain variable domain which specifically binds a second TAA and a first heavy chain variable domain and a first light chain variable domain which specifically binds human 4Ig-B7H3. In some embodiments, the bispecific antibody comprises antigen binding fragments, wherein the antigen-binding fragment can be a Fab, F(ab′)₂, Fv, a single chain Fv (scFv), or a single domain antibody.

In one embodiment, the multispecific antibody of the present disclosure binds to a second human TAA and/or human 4Ig-B7H3 with a binding affinity (K_{D}) of from 1×10⁻⁶ M to 1×10⁻¹⁰ M, or even 1×10⁻¹¹ M. In another embodiment, the multispecific antibody of the present disclosure binds to a second human TAA and/or human 4Ig-B7H3 with a binding affinity (K_{D}) of about 1×10⁻⁶ M, about 1×10⁻⁷ M, about 1×10⁻⁸ M, about 1×10⁻⁹ M, about 1×10⁻¹⁰ M, or about 1×10⁻¹¹ M.

In one embodiment, the present disclosure provides for a multispecific antibody or antigen-binding fragment thereof, wherein the first antigen binding domain that specifically binds to human 4Ig-B7H3 comprises:
(i) a heavy chain variable region (VH) that comprises (a) a HCDR1 (Heavy Chain Complementarity Determining Region 1) of SEQ ID NO: 11, (b) a HCDR2 of SEQ ID NO: 2 and (c) a HCDR3 of SEQ ID NO: 14 and a light chain variable region (VL) that comprises: (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region that comprises (a) a HCDR1 of SEQ ID NO: 1, (b) a HCDR2 of SEQ ID NO: 2 and (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6;
(iii) a heavy chain variable region that comprises (a) a HCDR1 of SEQ ID NO: 11, (b) a HCDR2 of SEQ ID NO: 2 and (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6;
(iv) a heavy chain variable region that comprises (a) a HCDR1 of SEQ ID NO: 1, (b) a HCDR2 of SEQ ID NO: 2 and (c) a HCDR3 of SEQ ID NO: 14; and a light chain variable region that comprises: (d) a LCDR1 of SEQ ID NO: 4, (e) a LCDR2 of SEQ ID NO: 5, and (f) a LCDR3 of SEQ ID NO: 6;
(v) a heavy chain variable region that comprises (a) a HCDR1 of SEQ ID NO: 1, (b) a HCDR2 of SEQ ID NO: 2 and (c) a HCDR3 of SEQ ID NO: 17; and a light chain variable region that comprises: (d) a LCDR1 of SEQ ID NO: 4, (e) a LCDR2 of SEQ ID NO: 5, and (f) a LCDR3 of SEQ ID NO: 6;
(vi) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 20, (e) a LCDR2 of SEQ ID NO: 5, and (f) a LCDR3 of SEQ ID NO: 6;
(vii) a heavy chain variable region that comprises (a) a HCDR1 of SEQ ID NO: 1, (b) a HCDR2 of SEQ ID NO: 2 and (c) a HCDR3 of SEQ ID NO: 3; and a light chain variable region that comprises: (d) a LCDR1 of SEQ ID NO: 23, (e) a LCDR2 of SEQ ID NO: 5, and (f) a LCDR3 of SEQ ID NO: 6; or
(viii) a heavy chain variable region that comprises (a) a HCDR1 of SEQ ID NO: 11, (b) a HCDR2 of SEQ ID NO: 2 and (c) a HCDR3 of SEQ ID NO: 28; and a light chain variable region that comprises: (d) a LCDR1 of SEQ ID NO: 23, (e) a LCDR2 of SEQ ID NO: 5, and (f) a LCDR3 of SEQ ID NO: 6; and the second antigen binding domain specifically binds to a second human TAA.

In another embodiment, the present disclosure provides for a multispecific antibody or antigen-binding fragment thereof, wherein the first antigen binding domain that specifically binds to human 4Ig-B7H3 comprises:
(i) a heavy chain variable region comprising SEQ ID NO: 26, and a light chain variable region comprising SEQ ID NO: 24;
(ii) a heavy chain variable region comprising SEQ ID NO: 7, and a light chain variable region comprising SEQ ID NO: 8;
(iii) a heavy chain variable region comprising SEQ ID NO: 12, and a light chain variable region comprising SEQ ID NO: 8;
(iv) a heavy chain variable region comprising SEQ ID NO: 15, and a light chain variable region comprising SEQ ID NO: 8;
(v) a heavy chain variable region comprising SEQ ID NO: 18, and a light chain variable region comprising SEQ ID NO: 8;
(vi) a heavy chain variable region comprising SEQ ID NO: 7, and a light chain variable region comprising SEQ ID NO: 21;
(vii) a heavy chain variable region comprising SEQ ID NO: 7, and a light chain variable region comprising SEQ ID NO: 24; or
(viii) a heavy chain variable region comprising SEQ ID NO: 29, and a light chain variable region comprising SEQ ID NO: 24, and the second antigen binding domain specifically binds to a second human TAA.

Previous experimentation (Coloma and Morrison Nature Biotech. 15:159-163 (1997)) described a tetravalent bispecific antibody which was engineered by fusing DNA encoding a single chain anti-dansyl antibody Fv (scFv) after the C terminus (CH3-scFv) or after the hinge (hinge-scFv) of an IgG3 anti-dansyl antibody. The present disclosure provides multivalent antibodies (e.g., tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains, which specifically bind at least two antigens.

In one embodiment, the multispecific antibody is a bispecific antibody. In another embodiment, the multispecific antibody further comprises an amino acid linker described herein, such as any one of SEQ ID NO: 35 to SEQ ID NO: 77.

III. Anti-Human 4Ig-B7H3 Antibodies Conjugated with a Cytotoxin

The anti-human 4Ig-B7H3 antibodies can be used to construct antibody drug conjugate (ADC). In one embodiment, the antibody or antigen-binding fragment thereof is conjugated to a cytotoxin. In another embodiment, the antibody or antigen-binding fragment thereof is conjugated to a cytotoxin via a cytotoxin linker.

### Cytotoxin

Cytotoxin or cytotoxic agents include any agent that is detrimental to the growth, viability or propagation of cells, including, but not limited to, tubulin-interacting agents and DNA-damaging agents. Examples of suitable cytotoxic agents and chemotherapeutic agents that can be conjugated to the antibodies of the present disclosure include, e.g., 1-(2chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), auristatins, bleomycin, busulfan, butyric acid, calicheamicins (e.g., calicheamicin gamma1), camptothecin, carminomycins, carmustine, cemadotins, cisplatin, colchicin, combretastatins, cyclophosphamide, cytarabine, cytochalasin B, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing.

### Cytotoxin Linker

Cytotoxin linkers or linkers for ADC are any group or moiety that links, connects, or bonds the antibody or antigen-binding proteins described herein with a therapeutic moiety, e.g., cytotoxic agent. Suitable linkers may be found, for example, in Antibody-Drug Conjugates and Immunotoxins; Phillips, G. L, Ed.; Springer Verlag: New York, 2013; Antibody-Drug Conjugates; Ducry, L, Ed.; Humana Press, 2013; Antibody-Drug Conjugates; Wang, J., Shen, W.-C, and Zaro, J. L, Eds.; Springer International Publishing, 2015, the contents of each incorporated herein in their entirety by reference.

Suitable binding agent or cytotoxin linkers for the antibody conjugates described herein are those that are sufficiently stable to exploit the circulating half-life of the antibody and, at the same time, capable of releasing its payload after antigen-mediated internalization of the conjugate. Linkers can be cleavable or non-cleavable. Cleavable linkers include linkers that are cleaved by intracellular metabolism following internalization, e.g., cleavage via hydrolysis, reduction, or enzymatic reaction. Non-cleavable linkers include linkers that release an attached payload via lysosomal degradation of the antibody following internalization. Suitable linkers include, but are not limited to, acid-labile linkers, hydrolysis-labile linkers, enzymatically cleavable linkers, reduction labile linkers, self-immolative linkers, and non-cleavable linkers. Suitable linkers also include, but are not limited to, those that are or comprise peptides, glucuronides, succinimide-thioethers, polyethylene glycol (PEG) units, hydrazones, mal-caproyl units, dipeptide units, valine-citruline units, and para-aminobenzyl (PAB) units.

Any cytotoxin linker molecule or linker technology known in the art can be used to create or construct an ADC of the present disclosure. In certain embodiments, the cytotoxin linker is a cleavable linker. According to other embodiments, the linker is a non-cleavable linker. Exemplary linkers that can be used in the context of the present disclosure include, linkers that comprise or consist of e.g., GGFG, MC (6-maleimidocaproyl), MP (maleimidopropanoyl), val-cit (valine-citrulline), val-ala (valine-alanine), dipeptide site in protease-cleavable linker, ala-phe (alanine-phenylalanine), dipeptide site in protease-cleavable linker, PAB (p-aminobenzyloxycarbonyl), SPP (N-Succinimidyl 4-(2-pyridylthio) pentanoate), SMCC (N-Succinimidyl 4-(N-maleimidomethyl)cyclohexane-1 carboxylate), SIAB (N-Succinimidyl (4-iodo-acetyl)aminobenzoate), and variants and combinations thereof. Additional examples of linkers that can be used in the context of the present disclosure are provided, e.g., in U.S. Pat. No. 7,754,681 and in Ducry, Bioconjugate Chem., 2010, 27:5-13, and the references cited therein, the contents of which are incorporated by reference herein in their entireties.

In certain embodiments, the cytotoxin linkers are stable in physiological conditions. In certain embodiments, the linkers are cleavable, for instance, able to release at least the payload portion in the presence of an enzyme or at a particular pH range or value. In some embodiments, a linker comprises an enzyme-cleavable moiety. Illustrative enzyme-cleavable moieties include, but are not limited to, peptide bonds, ester linkages, hydrazones, and disulfide linkages. In some embodiments, the linker comprises a cathepsin-cleavable linker.

In some embodiments, the cytotoxin linker comprises a non-cleavable moiety.

Suitable cytotoxin linkers also include, but are not limited to, those that are chemically bonded to two cysteine residues of a single binding agent, e.g., antibody. Such linkers can serve to mimic the antibody's disulfide bonds that are disrupted as a result of the conjugation process.

In some embodiments, the cytotoxin linker comprises one or more amino acids. Suitable amino acids include natural, non-natural, standard, non-standard, proteinogenic, non-proteinogenic, and L- or D-amino acids. In some embodiments, the cytotoxin linker comprises alanine, valine, glycine, leucine, isoleucine, methionine, tryptophan, phenylalanine, proline, serine, threonine, cysteine, tyrosine, asparagine, glutamine, aspartic acid, glutamic acid, lysine, arginine, histidine, or citrulline, a derivative thereof, or combination thereof. In certain embodiments, one or more side chains of the amino acids is linked to a side chain group, described below. In some embodiments, the linker comprises valine and citrulline. In some embodiments, the cytotoxin linker comprises lysine, valine, and citrulline. In some embodiments, the linker comprises lysine, valine, and alanine. In some embodiments, the linker comprises valine and alanine.

### IV. Fusion Protein Targeting Human 4Ig-B7H3

The anti-human 4Ig-B7H3 antibodies can be used to fuse with other proteins or other functional domains to form fusion proteins or chimeric proteins.

In some embodiments, the anti-human 4Ig-B7H3 antibodies are fused with immune checkpoints, immune stimulators, cytokines or a second TAA either directly or indirectly via an amino acid linker described herein.

In one embodiment, the anti-human 4Ig-B7H3 antibodies are fused with functional domains or receptor subunits that might serve to transduce the signal from the scFv and confer antibody specificity to immune cells, such as T cells, NK cells as well as other effector cells.

In some embodiments, the anti-human 4Ig-B7H3 antibodies are used to a construct chimeric antigen receptor (CAR). Detailed information about CAR constructs could be found in Guedan et al., (2019) Mol. Ther. Methods Clin. Dev. 12:145-156.

The functional domains or receptor subunits comprises transmembrane domain, hinge region, intracellular signaling domain, co-stimulatory domain, etc.

### V. Other Modifications

### Constant Region and Fc Region

The heavy chain constant region could be the wild type sequences of heavy chain constant region from the subclass of IgG1, IgG2, IgG3, or IgG4. The light chain constant region could be the wild type sequences of light chain from kappa or lambda type. In one embodiment, the heavy chain constant region is the wild type sequence of constant region from IgG1. The light chain constant region is the wild type sequence of the light chain from the kappa chain. In one embodiment, the heavy chain constant region has the amino acid sequence of SEQ ID NO: 20 and the light chain constant region has the amino acid sequence of SEQ ID NO: 21. In another embodiment, the heavy chain constant region has the amino acid sequence of SEQ ID NO: 1 comprising mutations E116P, L117A, L118A, G119del, and P212A. These amino acid mutations correspond to E233P, L234A, L235A, G236del, and P329A in EU numbering.

In one embodiment, the Fc region could be wild type Fc region of the subclass of IgG1, IgG2, IgG3, or IgG4. In one embodiment, the antibody or antigen-binding fragment thereof comprises a Fc domain of IgG1 or IgG4 with reduced effector function.

In one embodiment, the antibody or antigen-binding fragment thereof comprises a Fc domain with extended half-life. In another embodiment, the antibody or antigen-binding fragment thereof comprises a Fc domain of IgG1, wherein YTE mutation (M252Y/S254T/T256E, EU numbering, as described in U.S. Pat. No. 7,658,921) located at CH2 of IgG Fc region were introduced.

In another embodiment, antibodies of the present disclosure have strong Fc-mediated effector functions, and the antibodies mediate antibody-dependent cellular cytotoxicity (ADCC) against target cells.

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., (2009) MAbs. 1:332-338.

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see, Shields et al., (2001) J. Biol. Chem. 276:6591-6604).

In still another aspect, the glycosylation of the antibody is modified. For example, an aglycosylated antibody can be made (i.e., the antibody lacks or has reduced glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lec13 cells, with reduced ability to attach fucose to Asn (297)-linked carbohydrates, also resulting in hypofucosylation of antibodies expressed in that host cell (see also, Shields et al., (2002) J. Biol. Chem. 277:26733-26740). WO99/54342 by Umana et al., describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also, Umana et al., (1999) Nat. Biotech. 17:176-180).

In another aspect, if a reduction of ADCC is desired, human antibody subclass IgG4 was shown in many previous reports to have only modest ADCC and almost no CDC effector function (Moore G L, et al., (2010) Mabs. 2:181-189). However, natural IgG4 was found less stable in stress conditions such as in acidic buffer or under increasing temperature (Angal, S. (1993) Mol. Immunol. 30:105-108; Dall'Acqua, W. et al., (1998) Biochemistry. 37:9266-9273; Aalberse et al., (2002) Immunol. 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., (2007) Science. 317:1554-157). The mutation of serine to proline at position 228 (EU numbering system) appeared inhibitory to the IgG4 heavy chain separation (Angal, S. (1993) Mol. Immunol. 30:105-108; Aalberse et al., (2002) Immunol. 105:9-19). Some of the amino acid residues in the hinge and γFc region were reported to have impact on antibody interaction with Fcγ receptors (Chappel S M, et al., (1991) Proc. Natl. Acad. Sci. USA. 88:9036-9040; Mukherjee, J. et al., (1995) FASEB J. 9:115-119; Armour, K. L. et al., (1999) Eur. J. Immunol. 29:2613-2624; Clynes, R. A. et al, (2000) Nature Medicine. 6:443-446; Arnold J. N., (2007) Annu. Rev. Immunol. 25:21-50). Furthermore, some rarely occurring IgG4 isoforms in the human population can also elicit different physicochemical properties (Brusco, A. et al., (1998) Eur. J. Immunogenet. 25:349-55; Aalberse et al., (2002) Immunol. 105:9-19). To generate multispecific antibodies with low ADCC and CDC but with good stability, it is possible to modify the hinge and Fc region of human IgG4 and introduce a number of alterations. These modified IgG4 Fc molecules can be found in SEQ ID NOs: 83-88 of U.S. Pat. No. 8,735,553 to Li et al.

In another embodiment, the antibody of the present disclosure comprises Fc domain of human IgG4 with S228P and/or R409K substitutions (according to EU numbering system).

### Amino Acid Linkers

It is understood that whether there is an amino acid linker has minimal influence on the activity of the antibody or protein of the present disclosure.

It is also understood that the domains and/or regions of the polypeptide chains of the antibody or protein can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1. Such linker region may comprise a random assortment of amino acids, or a restricted set of amino acids. Such linker regions can be flexible or rigid (see US2009/0155275).

Multispecific antibodies have been constructed by genetically fusing two single chain Fv (scFv) or Fab fragments with or without the use of flexible linkers (Mallender et al., (1994) J. Biol. Chem. 269:199-206; Macket al., (1995) Proc. Natl. Acad. Sci. USA. 92:7021-5; Zapata et al., (1995) Protein Eng. 8.1057-62), via a dimerization device such as leucine Zipper (Kostelny et al., (1992) J. Immunol. 148:1547-53; de Kruifetal J. (1996) Biol. Chem. 271:7630-4) and Ig C/CH1 domains (Muller et al., (1998) FEBS Lett. 422:259-64); by diabody (Holliger et al., (1993) Proc. Nat. Acad. Sci. USA. 1998 90:6444-8; Zhu et al., (1996) Bio/Technology (NY). 14:192-6); Fab-scFv fusion (Schoonjans et al., (2000) J. Immunol. 165:7050-7); and mini antibody formats (Packet al., (1992) Biochemistry 31:1579-84; Packet al., (1993) Bio/Technology. 11:1271-7).

The antibodies or proteins as disclosed herein comprise an amino acid linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its antigen binding domains, CL domains, CH1 domains, Hinge region, CH2 domains, CH3 domains, or Fc regions. In some embodiments, the amino acids glycine and serine are comprised within the linker region. In another embodiment, the linker can be GS (SEQ ID NO: 35), GGS (SEQ ID NO: 36), GSG (SEQ ID NO: 37), SGG (SEQ ID NO: 38), GGG (SEQ ID NO: 39), GGGS (SEQ ID NO: 40), SGGG (SEQ ID NO: 41), GGGGS (SEQ ID NO: 42), GGGGSGS (SEQ ID NO: 43), GGGGSGS (SEQ ID NO: 44), GGGGSGGS (SEQ ID NO: 45), GGGGSGGGGS (SEQ ID NO: 46), GGGGSGGGGSGGGGS (SEQ ID NO: 47), AKTTPKLEEGEFSEAR (SEQ ID NO: 48), AKTTPKLEEGEFSEARV (SEQ ID NO: 49), AKTTPKLGG (SEQ ID NO: 50), SAKTTPKLGG (SEQ ID NO: 51), AKTTPKLEEGEFSEARV (SEQ ID NO: 52), SAKTTP (SEQ ID NO: 53), SAKTTPKLGG (SEQ ID NO: 54), RADAAP (SEQ ID NO: 55), RADAAPTVS (SEQ ID NO: 56), RADAAAAGGPGS (SEQ ID NO: 57), RADAAAA (G₄S)₄ (SEQ ID NO: 58), SAKTTP (SEQ ID NO: 59), SAKTTPKLGG (SEQ ID NO: 60), SAKTTPKLEEGEFSEARV (SEQ ID NO: 61), ADAAP (SEQ ID NO: 62), ADAAPTVSIFPP (SEQ ID NO: 63), TVAAP (SEQ ID NO: 64), TVAAPSVFIFPP (SEQ ID NO: 65), QPKAAP (SEQ ID NO: 66), QPKAAPSVTLFPP (SEQ ID NO: 67), AKTTPP (SEQ ID NO: 68), AKTTPPSVTPLAP (SEQ ID NO: 69), AKTTAP (SEQ ID NO: 70), AKTTAPSVYPLAP (SEQ ID NO: 71), ASTKGP (SEQ ID NO: 72), ASTKGPSVFPLAP (SEQ ID NO: 73), GENKVEYAPALMALS (SEQ ID NO: 74), GPAKELTPLKEAKVS (SEQ ID NO: 75), and GHEAAAVMQVQYPAS (SEQ ID NO: 76), GGGGSGGGGSGGGGSGGGGS (SEQ ID NO: 77) or any combination thereof (see, WO2007/024715).

### Dimerization Specific Amino Acids

In one embodiment, the multivalent antibody comprises at least one dimerization specific amino acid change. The dimerization specific amino acid changes result in “knobs into holes” interactions, and increases the assembly of correct multivalent antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551. The dimerization specific amino acids can also be within the Fc domain and can be in combination with dimerization specific amino acids within the CH1 or CL domains. In one embodiment, the disclosure provides a bispecific antibody comprising at least one dimerization specific amino acid pair.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies or proteins described herein, e.g., polynucleotides encoding heavy chain variable regions or light chain variable regions comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from SEQ ID NO: 9, SEQ ID NO: 13, SEQ ID NO: 16, SEQ ID NO: 19, SEQ ID NO: 27, or SEQ ID NO: 30. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from SEQ ID NO: 10, SEQ ID NO: 22, or SEQ ID NO: 25.

The present disclosure also provides polynucleotides encoding the scFv described herein, e.g., the polynucleotides encoding the amino acid sequences of SEQ ID NO: 32.

The polynucleotides of the present disclosure can encode the variable region sequence of the multispecific antibody described herein. They can also encode both a variable region and a constant region of the multispecific antibody. In another embodiment, the polynucleotides of the present disclosure can encode the amino acid sequences of fusion proteins described herein.

In some embodiments, the polynucleotides described herein could be codon-optimized for expression in host cells, e.g., eukaryotic cells, more specifically mammalian cells (e.g., CHO cells).

Also provided in the present disclosure are expression vectors and host cells for producing the antibodies herein. The choice of expression vector depends on the intended host cells in which the vector is to be expressed. Typically, the expression vectors contain a promoter and other regulatory sequences (e.g., enhancers) that are operably linked to the polynucleotides encoding antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., (1994) Results Probl. Cell Differ. 20:125; Bittner et al., (1987) Meth. Enzymol., 153:516). For example, the SV40 enhancer or CMV enhancer can be used to increase expression in mammalian host cells.

The host cells for harboring and expressing the antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibodies. Insect cells in combination with baculovirus vectors can also be used. In other aspects, mammalian host cells are used to express and produce the antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., (1986) Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of 4Ig-B7H3. In one aspect, the antibodies or antigen-binding fragments are useful for detecting the presence of 4Ig-B7H3 in a biological sample. The term “detecting” as used herein includes quantitative or qualitative detection. In certain aspects, a biological sample comprises a cell or tissue. In other aspects, such tissues include normal and/or cancerous tissues that express 4Ig-B7H3 at higher levels relative to other tissues.

In one aspect, the present disclosure provides a method of detecting the presence of 4Ig-B7H3 in a biological sample. In certain aspects, the method comprises contacting the biological sample with an anti-4Ig-B7H3 antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of 4Ig-B7H3. In certain aspects, the method comprises contacting a test cell with an anti-4Ig-B7H3 antibody; determining the level of expression (either quantitatively or qualitatively) of 4Ig-B7H3 expressed by the test cell by detecting binding of the anti-4Ig-B7H3 antibody to the 4Ig-B7H3 polypeptide; and comparing the level of expression by the test cell with the level of 4Ig-B7H3 expression in a control cell (e.g., a normal cell of the same tissue origin as the test cell or a non-4Ig-B7H3 expressing cell), wherein a higher level of 4Ig-B7H3 expression in the test cell as compared to the control cell indicates the presence of a disorder associated with expression of 4Ig-B7H3.

### Methods of Treatment

The antibodies or antigen-binding fragments of the present disclosure are useful in a variety of applications including, but not limited to, methods for the treatment of a 4Ig-B7H3-associated disorder or disease. In one aspect, the 4Ig-B7H3-associated disorder or disease is a cancer. In some embodiments, the cancer is 4Ig-B7H3 positive.

In one aspect, the present disclosure provides a method of treating cancer. In certain aspects, the method comprises administering to a patient in need an effective amount of an anti-4Ig-B7H3 antibody or antigen-binding fragment or 4Ig-B7H3 antibody containing multispecific antibody. In another aspect, the present disclosure provides anti-4Ig-B7H3 antibody or antigen-binding fragment or the multispecific antibody, or the pharmaceutical composition for use in the treatment of cancer. In another aspect, the present disclosure provides the use of the anti-4Ig-B7H3 antibody or antigen-binding fragment, multispecific antibody or antigen-binding fragment thereof, or the pharmaceutical composition in the manufacture of a medicament for the treatment of cancer.

The cancer can include, without limitation, colorectal carcinoma, prostate cancer, pancreatic cancer, breast cancer, ovarian cancer, kidney cancer, lung cancer, or esophageal carcinoma. In one embodiment, the lung cancer is non-small cell lung cancer (NSCLC) or small cell lung cancer (SCLC). In another embodiment, the non-small cell lung cancer is squamous non-small cell lung cancer. In another embodiment, the esophageal carcinoma is esophageal squamous cell carcinoma.

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules include but are not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician.

### Combination Therapy

In one aspect, anti-4Ig-B7H3 antibodies or anti-4Ig-B7H3 antibody containing multispecific antibodies of the present disclosure can be used in combination with other therapeutic agents. Other therapeutic agents that can be used with the 4Ig-B7H3 antibodies of the present disclosure include but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

Anti-4Ig-B7H3 antibodies of the present disclosure can be used in combination with other therapeutics, for example, other immune checkpoint antibodies. Such immune checkpoint antibodies can include anti-PD1 antibodies. Anti-PD1 antibodies can include, without limitation, Tislelizumab, Pembrolizumab or Nivolumab. Tislelizumab (SEQ ID Nos: 22 and 23 in Table 7) is disclosed in U.S. Pat. No. 8,735,553. Pembrolizumab (formerly MK-3475), is disclosed in U.S. Pat. Nos. 8,354,509 and 8,900,587 and is a humanized IgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human IgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

In one embodiment, the present disclosure provides a use of the combination of the anti-4Ig-B7H3 antibodies or anti-4Ig-B7H3 antibody containing multispecific antibody and anti-PD-1 antibody (such as Tislelizumab or other anti-PD-1 antibody mentioned above) in the manufacture of a medicament for the treatment of cancer, such as the cancers mentioned above. In another embodiment, the present disclosure provides the combination of the anti-4Ig-B7H3 antibodies or anti-4Ig-B7H3 antibody containing multispecific antibody and anti-PD-1 antibody (such as Tislelizumab or other anti-PD-1 antibody mentioned above) for use in the treatment of cancer, such as the cancers mentioned above.

The combination therapy is intended to mean, and does refer to and include any one of the following:
simultaneous administration of such combination therapy to a patient in need of treatment, when such components are formulated together into a single dosage form which releases said components at substantially the same time to said patient,
substantially simultaneous administration of such combination to a patient in need of treatment, when such components are formulated apart from each other into separate dosage forms which are taken at substantially the same time by said patient, whereupon said components are released at substantially the same time to said patient,
sequential administration of such combination therapy to a patient in need of treatment, when such components are formulated apart from each other into separate dosage forms which are taken at consecutive times by said patient with a significant time interval between each administration, whereupon said components are released at substantially different times to said patient; and sequential administration of such combination to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

### Pharmaceutical Compositions

Also provided are compositions, including pharmaceutical formulations, comprising an anti-4Ig-B7H3 antibody or antigen-binding fragment thereof (including multispecific antibodies), or polynucleotides comprising sequences encoding the antibody or antigen-binding fragment. In certain embodiments, compositions comprise one or more anti-4Ig-B7H3 antibodies or antigen-binding fragments, or one or more polynucleotides comprising sequences encoding one or more anti-4Ig-B7H3 antibodies or antigen-binding fragments. These compositions can further comprise suitable carriers, such as pharmaceutically acceptable excipients including buffers, which are well known in the art.

Pharmaceutical formulations of an anti-4Ig-B7H3 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g., films, or microcapsules.

The formulations to be used for in vivo administration are generally sterile. Sterility can be readily accomplished, e.g., by filtration through sterile filtration membranes.

### Definitions

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “human 4Ig-B7H3” refers to the 4Ig isoform of a type I transmembrane protein B7H3 in humans. In some embodiments, the amino acid sequence of the human 4Ig-B7H3 comprises SEQ ID NO: 80.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient comprising, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antibody” includes, but is not limited to, monoclonal antibodies, human antibodies, humanized antibodies, chimeric antibodies, and anti-idiotypic (anti-Id) antibodies, a human engineered antibody, a single chain antibody (scFv), a single domain antibody, a Fab fragment, a Fab′ fragment, or a F(ab′)₂ fragment. The antibodies can be of any isotype/class (e.g., IgG, IgE, IgM, IgD, IgA and IgY), or subclass (e.g., IgG1, IgG2, IgG3, IgG4, IgAQ1 and IgA2). In addition, the antibody includes the derivative agents thereof, such as fusion protein, multispecific antibody, or antibody-drug conjugate (ADC). In addition, the antibody includes the derivative agents thereof by linking to another agent (such as other drug or antibody) directly or indirectly or forming a complex with another agent.

The term “chimeric antibody” means molecules made up of domains from different species, i.e., fusing the variable domain of an antibody from one host species (e.g. mouse, rabbit, llama, etc.) with the constant domain of an antibody from a different species (e.g. human).

In some embodiments, the anti-4Ig-B7H3 antibodies comprise at least one antigen-binding site, at least a variable region. In some embodiments, the anti-4Ig-B7H3 antibodies comprise an antigen-binding fragment from an 4Ig-B7H3 antibody described herein. In some embodiments, the anti-4Ig-B7H3 antibody is isolated or recombinant. In some embodiments, the anti-4Ig-B7H3 antibodies also encompass multispecific antibodies targeting 4Ig-B7H3 as at least one arm and targeting other antigen(s) as another arm(s).

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., (1975) Nature. 256:495-497; U.S. Pat. No. 4,376,110; Ausubel et al., (1992) CURRENT PROTOCOLS IN MOLECULAR BIOLOGY; Harlow et al., (1988) ANTIBODIES: A LABORATORY MANUAL, Cold spring Harbor Laboratory; and Colligan et al., (1993) CURRENT PROTOCOLS IN IMMUNOLOGY. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as a, 8, &, Y, or u, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., (2001) Nucleic Acids Res. 29:205-206; Chothia and Lesk, (1987) J. Mol. Biol. 196:901-917; Chothia et al., (1989) Nature. 342:877-883; Chothia et al., (1992) J. Mol. Biol. 227:799-817; Al-Lazikani et al. (1997) J. Mol. Biol. 273:927-748 ImMunoGenTics (IMGT) numbering (Lefranc, M.-P., (1999) The Immunologist. 7, 132-136; Lefranc, M.-P. et al., (2003) Dev. Comp. Immunol. 27, 55-77 (“IMGT” numbering scheme)). Definitions of antigen combining sites are also described in the following: Ruiz et al., (2000) Nucleic Acids Res. 28:219-221; and Lefranc, M. P., (2001) Nucleic Acids Res. 29:207-209; MacCallum et al., (1996) J. Mol. Biol. 262:732-745; and Martin et al., (1989) Proc. Natl. Acad. Sci. USA, 86:9268-9272; Martin et al., (1991) Methods Enzymol. 203:121-153; and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). Sec, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196:901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

“Antigen-binding domain” as used herein, comprise at least six CDRs and specifically bind to an epitope (or three CDRs in terms of single domain antibody). An “antigen-binding domain” of a multispecific antibody (e.g., a bispecific antibody) comprises a first antigen binding domain that specifically binds to a first epitope and a second antigen binding domain specifically binds to a second epitope. Multispecific antibodies can be bispecific, trispecific, tetraspecific etc., with antigen binding domains directed to each specific epitope. Multispecific antibodies can be multivalent (e.g., a bispecific tetravalent antibody) that comprises multiple antigen binding domains, for example, 2, 3, 4 or more antigen binding domains that specifically bind to a first epitope and 2, 3, 4 or more antigen binding domains that specifically bind a second epitope.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine, rabbit, llama, etc.) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent/camelid antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., (2000) J. Mol. Biol. 296:57-86.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to 4Ig-B7H3. Specifically, common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H} interfaces or V_{H}/V_{L} interfaces of antibodies (see, e.g., US 2011/0287009, US2007/0178552, WO 96/027011, WO 98/050431, and Zhu et al., (1997) Protein Science. 6:781-788). In some embodiments, knob-into-holes insure the correct pairing of two different heavy chains together during the manufacture of multispecific antibodies. For example, multispecific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also insure correct pairing.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al, (1977) Nuc. Acids Res. 25:3389-3402; and Altschul et al., (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-4Ig-B7H3 multispecific antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-4IG-B7H3 antibody is administered to the subject at the same time as, just before, or just after administration of an additional therapeutic agent. In certain embodiments, an anti-4Ig-B7H3 antibody is administered as a co-formulation with an additional therapeutic agent.

### Equivalent

It is to be understood that while the present invention has been described in conjunction with the detailed description thereof, the foregoing description is intended to illustrate and not limit the scope of the invention, which is defined by the scope of the appended claims. Other aspects, advantages, and modifications are within the scope of the following claims.

It is to be understood that one, some, any or all of the features of the various embodiments described herein may be combined to form further embodiments of the present disclosure. These and other aspects of the present disclosure will become apparent to those skilled in the art.

### EXAMPLES

### Example 1. Generation of Mouse Anti-B7H3 Monoclonal Antibodies

### Immunization

To generate antibodies against B7H3, one cohort of 5 transgenic mice, engineered to produce human variant regions and rat constant regions, were immunized with extracellular binding domain (ECD) of human 4Ig-B7H3 antigen with each cohort being subjected to an immunization strategy comprising a unique combination of human B7H3 antigen, dose, injection route, adjuvant. A total of 5 animals in 1 cohort were immunized. Animals received immunizations over varying periods between 0 and 100 days. To monitor immune responses, titrated serum was screened by ELISA, typically after 30-100 days of 2-6 immunizations. Serum was screened for antibody (Ab) binding to B7H3 antigen. B7H3-specific antibody responses were measured in each animal, and animals with sufficient titers of anti-B7H3 were selected for final boost for 4 days.

### Hybridoma Fusion and Screening

Lymphoid organs, including spleens and lymph nodes, were isolated from mice immunized as described above. Hybridomas were generated by fusions with immortalized mouse myeloma cells derived from the SP2/0 by PEG based fusion. The resulting cells were plated in 96 well cell culture plates using regular 1640 medium (Gibco™) supplemented with HAT for selection of hybridomas. After 10-13 days of culture and growth media replacement, hybridoma culture supernatants were collected from individual wells and screened to identify wells with secreted B7H3-specific antibodies. All supernatants were initially screened against human ECD-4Ig-B7H3-hFc. Antibodies binding to human ECD-4Ig-B7H3-hFc were measured through ELISA. Supernatants from 5 hybridoma fusions were screened for B7H3 antibodies. Briefly, 2 μg/mL human ECD-4Ig-B7H3-hFc was coated in the 96 well ELISA plates, and 50 μl of hybridoma culture supernatant was co-incubated with human ECD-4Ig-B7H3-hFc for 30-60 minutes. It was then washed and incubated with anti-rat IgG Fc secondary Ab conjugated to HRP. After incubation and washing, and the plates were developed with HRP substrate and absorbance was measured.

Hybridomas from positive wells were transferred to 24-well plates with fresh culture media to grow for 2-3 days before screening again by flow cytometry to confirm antibody binding to human 4Ig-B7H3 and cyno B7H3 overexpressing cell lines.

Antibodies binding to human B7H3 overexpressing cells and cyno B7H3 overexpressing cells were measured through FACS respectively. Briefly, 100 μl of hybridoma culture supernatant and human 4Ig-B7H3 or cyno B7H3 overexpressing cells were co-incubated for 30-60 minutes, washed, and incubated with anti-IgG Fc secondary Ab conjugated to APC. After incubation and washing, fluorescence was measured by flow cytometry.

### Subcloning and Sequence Analysis

Selected anti-B7H3 Ab-secreting hybridomas were subcloned once or twice to ensure monoclonality. Briefly, the positive hybridoma clones were sub-cloned by limiting dilution. After 7-10 days, culture supernatant was screened by ELISA and flow cytometry was conducted as previously described to confirm human and cyno B7H3 antigen binding. Stable hybridoma subclones were cultured in vitro to cell cryopreservation and antibody VH and VL gene cloning and sequencing.

The anti-B7H3 Ab-secreting hybridomas after subcloning were lysed by lysis buffer. The mRNA containing lysates were subsequently transferred to 96-well well plates for mRNA isolation, cDNA synthesis and DNA sequencing using Super Script III first-strand synthesis SuperMix™ (Invitrogen) according to the manufacturer's instructions.

The sequences of resulting human antibody BGA-3726 are listed on Table 7.

### Example 2. Binding Kinetics and Affinity Determination of Anti-B7H3 Antibodies by SPR

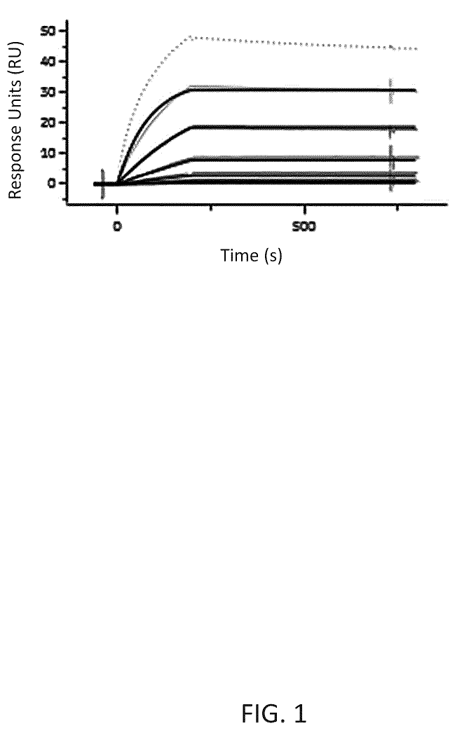

Anti-B7H3 mAb BGA-3726 was purified and characterized for the binding kinetics by SPR assays using BIAcore™ T-200 (GE Life Sciences). Briefly, anti-mouse IgG Fc antibody was immobilized on an activated CM5 biosensor chip (Cat. No. BR100530, GE Life Sciences). Purified antibodies were flowed over the chip surface and captured by anti-mouse IgG Fc antibody. Then a serial dilution of human ECD-4Ig-B7H3-hFc was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of anti-B7H3 mAbs are shown in Table 1 and FIG. 1.

TABLE 1
Binding kinetics of BGA-3726

| Abs# | Antigen | ka (1/Ms) | kd (1/s) | KD (M) |
|---|---|---|---|---|
| BGA-3726 | Human B7H3 | 3.63E+05 | 4.49E−06 | 1.24E−11 |

### Example 3. Determining the Binding Affinity of Anti-B7H3 Antibodies to B7H3 Expressed on Stable Cell Lines

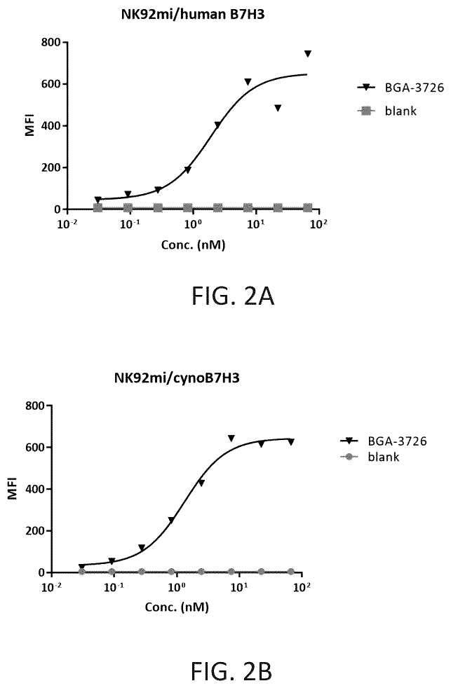

The binding affinity of anti-B7H3 mAb to human 4Ig-B7H3 and cyno B7H3 overexpressing cell lines were determined by FACS. Briefly, human or cyno B7H3 overexpressing cells were incubated with serially diluted purified antibodies, washed, and incubated with anti-mouse IgG secondary Ab conjugated to APC. After incubation and washing, fluorescence was measured by flow cytometry. The binding affinity profiles of anti-B7H3 mAbs are shown in Tables 2-3 and FIGS. 2A-B.

TABLE 2
Cellular binding affinity of BGA-3276 with human B7-H3

| Abs# | Cell line | EC50 (nM) |
|---|---|---|
| BGA-3726 | Human B7H3 | 1.91 |

TABLE 3
Cellular binding affinity of BGA-3276 with cyno B7-H3

| Abs# | Cell line | EC50 (nM) |
|---|---|---|
| BGA-3726 | Cyno B7H3 | 1.32 |

### Example 4. Determination of the Non-Specific Binding of Anti-B7H3 Antibodies

Non-specific binding activity of anti-B7H3 mAb BGA-3276 were determined by FACS. Briefly, Daudi cells which do not express B7H3 were incubated with purified BGA-3276, washed, and incubated with anti-rat IgG secondary Ab conjugated to APC. After incubation and washing, fluorescence was measured by flow cytometry. The binding MFI is shown in Table 4. BGA-3276 did not show any non-specific binding to B7H3 negative cells.

TABLE 4
MFI of BGA-3276 binding with Daudi

| Abs# | MFI of NK92mi |
|---|---|
| BGA-3726 | 4.15 |
| mIgG | 16.00 |

### Example 5. Engineering of the Anti-Human B7H3 mAb BGA-3726

BGA-3726 (SEQ ID NOs: 1-10) is a human antibody which is comprised of fully human Vh and Vk regions. To improve its developability, BGA-3726 was re-engineered to remove potential post translational modifications (PTMs) and optimize the biophysical stability for therapeutics use in humans.

BGA-3726 and antibodies engineered from BGA-3726 were constructed as human full-length antibody format using in-house developed expression vectors that contain constant regions of a human IgG1 variant (SEQ ID NO: 31) and kappa chain, respectively, with easy adapting sub-cloning sites. Expression and preparation of BGA-3726 and engineered antibodies thereof was achieved by co-transfection of the heavy chain and corresponding light chain constructs into 293G cells (developed in house) and by purification using a Protein A column. The purified antibodies were concentrated to 0.5-5 mg/mL in PBS and stored in aliquots in −80° C. freezer.

Based on the BGA-3726 original clone, several single-mutations in the CDRs were made to remove potential post-translation modification (PTM) sites. The potential PTM sites included potential oxidation sites W34, W96 and W98 (Kabat numbering) in the HCDRs and potential deamidation site N29 (NG) in LCDR1. All mutations were made using primers containing mutations at specific positions and a site directed mutagenesis kit (Cat. No. FM111-02, TransGen, Beijing, China). The desired mutations were verified by sequencing analysis. These BGA-3726 variant antibodies BGA-4826 comprising W34Y in HCDR1 (SEQ ID Nos: 11, 2-6, 12, 8, 13, 10), BGA-4498 comprising W96Y in HCDR3 (SEQ ID Nos: 1, 2, 14, 4-6, 15, 8, 16, 10), BGA-4269 comprising W98Y in HCDR3 (SEQ ID Nos: 1, 2, 17, 4-6, 18, 8, 19, 10), BGA-4380 comprising N29Q in LCDR1 (SEQ ID Nos: 1-3, 20, 5-6, 7, 21, 9, 22) and BGA-4265 comprising G30A in LCDR1 (SEQ ID Nos: 1-3, 23, 5-6, 7, 24, 9, 25) were tested in SPR binding assays.

Taken together, the engineered versions of humanized monoclonal antibodies, BGA-6938 (SEQ ID Nos: 11, 2, 14, 23, 5-6, 26, 24, 27, 25) comprising W34Y in HCDR1, W96Y in HCDR3 and G30A in LCDR1 based on BGA-3726 and BGA-5488 (SEQ ID Nos: 11, 2, 28, 23, 5-6, 29, 24, 30, 25) comprising W34Y in HCDR1, W96Y in HCDR3, W98Y in HCDR3 and G30A in LCDR1 based on BGA-3726, were derived from the mutation process described as above, and characterized in detail. The results showed that BGA-3726, BGA-6938 and BGA-5488 were comparable in binding affinity to recombinant B7H3.

For affinity determination, antibodies were captured by an anti-human Fc surface, and used in the affinity-assay based on surface plasmon resonance (SPR) technology. The results of SPR-determined binding profiles of anti-B7H3 antibodies to the ECD of human 4Ig-B7H3 (Sinobiological, Cat: 11188-H08H) are summarized in Table 5. BGA-6938 (SEQ ID NOs: 11, 2, 14, 23, 5-6, 26, 24, 27, 25) and BGA-5488 (SQ ID Nos: 11, 2, 28, 23, 5-6, 29, 24, 30, 25) have comparable binding affinities with dissociation constant at 0.443 nM and 1.11 nM, respectively, which are comparable to that of BGA-3726.

TABLE 5
Comparison of BGA-3726 and engineered antibodies
thereof on binding affinities to B7H3 by SPR

| | Test 1 | | | Test 2 | | |
|---|---|---|---|---|---|---|
| Ab | kₒₙ (M⁻¹s⁻¹) | k_{off} (s⁻¹) | K_{D} (M) | kₒₙ (M⁻¹s⁻¹) | k_{off} (s⁻¹) | K_{D} (M) |
| BGA-3726 | 3.21E+05 | 1.36E−04 | 4.24E−010 | 4.42E+05 | 2.07E−04 | 4.70E−010 |
| BGA-4826 | 2.88E+05 | 1.71E−04 | 5.96E−010 | — | — | — |
| BGA-4498 | 3.15E+05 | 1.99E−04 | 6.32E−010 | — | — | — |
| BGA-4269 | 1.42E+05 | 1.36E−04 | 4.24E−010 | — | — | — |
| BGA-4380 | 3.73E+05 | 2.37E−04 | 6.35E−010 | — | — | — |
| BGA-4265 | 4.14E+05 | 1.28E−04 | 3.08E−010 | — | — | — |
| BGA-6938 | — | — | — | 5.06E+04 | 2.24E−04 | 4.43E−010 |
| BGA-5488 | — | — | — | 2.95E+04 | 3.26E−04 | 1.11E−09 |

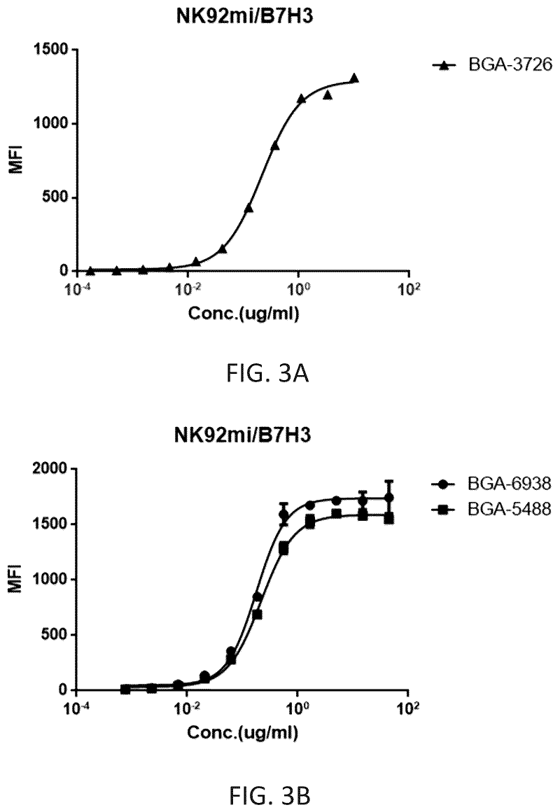

To evaluate the binding activity of BGA-6938 and BGA-5488 to bind native B7H3 on live cells, NK92mi cells were engineered to over-express human 4Ig-B7H3. Live NK92mi/B7H3 cells were seeded in 96-well plate and were incubated with a series of dilutions of BGA-3726, BGA-6938 and BGA-5488. Goat anti-human IgG was used as the second antibody to detect antibody binding to the cell surface. EC₅₀ values for dose-dependent binding to human native B7H3 were determined by fitting the dose-response data to the four-parameter logistic model with GraphPad Prism. As shown in FIG. 3A-3B and Table 6. The engineered antibodies BGA-6938 and BGA-5488 retained binding affinities of the parental clone BGA-3726 to native B7H3.

TABLE 6
Comparison on the EC50 of BGA-3726 and the humanized
antibodies thereof to NK92mi/B7H3 cells by FACS

NK92mi/B7H3 binding EC50 (nM)

| Antibody | Test1 | Test2 |
|---|---|---|
| BGA-3726 | 1.44 | — |
| BGA-6938 | — | 1.19 |
| BGA-5488 | — | 1.45 |

TABLE 7
Sequences of antibodies

| Antibody | SEQ ID NO | | SEQUENCE |
|---|---|---|---|
| BGA-3726 | SEQ ID NO: 1 | HCDR1 | TSNWWN |
| | | (Kabat) | |
| | SEQ ID NO: 2 | HCDR2 | EIYPTGNTNYSPSLKS |
| | | (Kabat) | |
| | SEQ ID NO: 3 | HCDR3 | GWNWFDP |
| | | (Kabat) | |
| | SEQ ID NO: 4 | LCDR1 | RTSQSINGILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 7 | VH | QVQLQESGPGLVKPSGTLSLTCAVSGGS |
| | | AA | ISTSNWWNWVRQSPGKGLEWIGEIYPTG |
| | | | NTNYSPSLKSRVTISIDKSKNQFSLKLNS |
| | | | VTAADTAVYYCARGWNWFDPWGQGT |
| | | | LVTVSS |
| | SEQ ID NO: 8 | VL | EIVLTQSPGTLSLSPGERATLSCRTSQS |
| | | AA | INGILLAWYQQKPGQAPRLLIYGASSRA |
| | | | TGIPDRFSGSGSGTDFTLSISRLEPEDF |
| | | | AVYYCQQYGSSPRTFGQGTKVEIK |
| | SEQ ID NO: 9 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTGGTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATGGAACTGGTTCGACCCCTGG |
| | | | GGCCAGGGAACCCTGGTCACCGTCTCC |
| | | | TCA |
| | SEQ ID NO: 10 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTAACGGCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| BGA-4826 | SEQ ID NO: 11 | HCDR1 | TSNYWN |
| | | (Kabat) | |
| | SEQ ID NO: 2 | HCDR2 | EIYPTGNTNYSPSLKS |
| | | (Kabat) | |
| | SEQ ID NO: 3 | HCDR3 | GWNWFDP |
| | | (Kabat) | |
| | SEQ ID NO: 4 | LCDR1 | RTSQSINGILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 12 | VH | QVQLQESGPGLVKPSGTLSLTCAVSGGS |
| | | AA | ISTSNYWNWVRQSPGKGLEWIGEIYPTG |
| | | | NTNYSPSLKSRVTISIDKSKNQFSLKLNS |
| | | | VTAADTAVYYCARGWNWFDPWGQGT |
| | | | LVTVSS |
| | SEQ ID NO: 8 | VL | EIVLTQSPGTLSLSPGERATLSCRTSQSIN |
| | | AA | GILLAWYQQKPGQAPRLLIYGASSRATG |
| | | | IPDRFSGSGSGTDFTLSISRLEPEDFAVYY |
| | | | CQQYGSSPRTFGQGTKVEIK |
| | SEQ ID NO: 13 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTACTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATGGAACTGGTTCGACCCCTGG |
| | | | GGCCAGGGAACCCTGGTCACCGTCTCC |
| | | | TCA |
| | SEQ ID NO: 10 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTAACGGCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| BGA-4498 | SEQ ID NO: 1 | HCDR1 | TSNWWN |
| | | (Kabat) | |
| | SEQ ID NO: 2 | HCDR2 | EIYPTGNTNYSPSLKS |
| | | (Kabat) | |
| | SEQ ID NO: 14 | HCDR3 | GYNWFDP |
| | | (Kabat) | |
| | SEQ ID NO: 4 | LCDR1 | RTSQSINGILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 15 | VH | QVQLQESGPGLVKPSGTLSLTCAVSGGS |
| | | AA | ISTSNWWNWVRQSPGKGLEWIGEIYPTG |
| | | | NTNYSPSLKSRVTISIDKSKNQFSLKLNS |
| | | | VTAADTAVYYCARGYNWFDPWGQGTL |
| | | | VTVSS |
| | SEQ ID NO: 8 | VL | EIVLTQSPGTLSLSPGERATLSCRTSQSIN |
| | | AA | GILLAWYQQKPGQAPRLLIYGASSRATG |
| | | | IPDRFSGSGSGTDFTLSISRLEPEDFAVYY |
| | | | CQQYGSSPRTFGQGTKVEIK |
| | SEQ ID NO: 16 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTGGTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATACAACTGGTTCGACCCCTGG |
| | | | GGCCAGGGAACCCTGGTCACCGTCTCC |
| | | | TCA |
| | SEQ ID NO: 10 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTAACGGCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| BGA-4269 | SEQ ID NO: 1 | HCDR1 | TSNWWN |
| | | (Kabat) | |
| | SEQ ID NO: 2 | HCDR2 | EIYPTGNTNYSPSLKS |
| | | (Kabat) | |
| | SEQ ID NO: 17 | HCDR3 | GWNYFDP |
| | | (Kabat) | |
| | SEQ ID NO: 4 | LCDR1 | RTSQSINGILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 18 | VH | QVQLQESGPGLVKPSGTLSLTCAVSGGS |
| | | AA | ISTSNWWNWVRQSPGKGLEWIGEIYPTG |
| | | | NTNYSPSLKSRVTISIDKSKNQFSLKLNS |
| | | | VTAADTAVYYCARGWNYFDPWGQGTL |
| | | | VTVSS |
| | SEQ ID NO: 8 | VL | EIVLTQSPGTLSLSPGERATLSCRTSQSIN |
| | | AA | GILLAWYQQKPGQAPRLLIYGASSRATG |
| | | | IPDRFSGSGSGTDFTLSISRLEPEDFAVYY |
| | | | CQQYGSSPRTFGQGTKVEIK |
| | SEQ ID NO: 19 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTGGTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATGGAACTACTTCGACCCCTGG |
| | | | GGCCAGGGAACCCTGGTCACCGTCTCC |
| | | | TCA |
| | SEQ ID NO: 10 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTAACGGCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| BGA-4380 | SEQ ID NO: 1 | HCDR1 | TSNWWN |
| | | (Kabat) | |
| | SEQ ID NO: 2 | HCDR2 | EIYPTGNTNYSPSLKS |
| | | (Kabat) | |
| | SEQ ID NO: 3 | HCDR3 | GWNWFDP |
| | | (Kabat) | |
| | SEQ ID NO: 20 | LCDR1 | RTSQSIQGILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 7 | VH | QVQLQESGPGLVKPSGTLSLTCAVSGGS |
| | | AA | ISTSNWWNWVRQSPGKGLEWIGEIYPTG |
| | | | NTNYSPSLKSRVTISIDKSKNQFSLKLNS |
| | | | VTAADTAVYYCARGWNWFDPWGQGT |
| | | | LVTVSS |
| | SEQ ID NO: 21 | VL | EIVLTQSPGTLSLSPGERATLSCRTSQSIQ |
| | | AA | GILLAWYQQKPGQAPRLLIYGASSRATG |
| | | | IPDRFSGSGSGTDFTLSISRLEPEDFAVYY |
| | | | CQQYGSSPRTFGQGTKVEIK |
| | SEQ ID NO: 9 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTGGTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATGGAACTGGTTCGACCCCTGG |
| | | | GGCCAGGGAACCCTGGTCACCGTCTCC |
| | | | TCA |
| | SEQ ID NO: 22 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTCAGGGCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| BGA-4265 | SEQ ID NO: 1 | HCDR1 | TSNWWN |
| | | (Kabat) | |
| | SEQ ID NO: 2 | HCDR2 | EIYPTGNTNYSPSLKS |
| | | (Kabat) | |
| | SEQ ID NO: 3 | HCDR3 | GWNWFDP |
| | | (Kabat) | |
| | SEQ ID NO: 23 | LCDR1 | RTSQSINAILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 7 | VH | QVQLQESGPGLVKPSGTLSLTCAVSGGS |
| | | AA | ISTSNWWNWVRQSPGKGLEWIGEIYPTG |
| | | | NTNYSPSLKSRVTISIDKSKNQFSLKLNS |
| | | | VTAADTAVYYCARGWNWFDPWGQGT |
| | | | LVTVSS |
| | SEQ ID NO: 24 | VL | EIVLTQSPGTLSLSPGERATLSCRTSQSIN |
| | | AA | AILLAWYQQKPGQAPRLLIYGASSRATG |
| | | | IPDRFSGSGSGTDFTLSISRLEPEDFAVYY |
| | | | CQQYGSSPRTFGQGTKVEIK |
| | SEQ ID NO: 9 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTGGTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATGGAACTGGTTCGACCCCTGG |
| | | | GGCCAGGGAACCCTGGTCACCGTCTCC |
| | | | TCA |
| | SEQ ID NO: 25 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTAACGCCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| BGA-6938 | SEQ ID NO: 11 | HCDR1 | TSNYWN |
| | | (Kabat) | |
| | SEQ ID NO: 2 | HCDR2 | EIYPTGNTNYSPSLKS |
| | | (Kabat) | |
| | SEQ ID NO: 14 | HCDR3 | GYNWFDP |
| | | (Kabat) | |
| | SEQ ID NO: 23 | LCDR1 | RTSQSINAILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 26 | VH | QVQLQESGPGLVKPSGTLSLTCAVSGGS |
| | | AA | ISTSNYWNWVRQSPGKGLEWIGEIYPTG |
| | | | NTNYSPSLKSRVTISIDKSKNQFSLKLNS |
| | | | VTAADTAVYYCARGYNWFDPWGQGTL |
| | | | VTVSS |
| | SEQ ID NO: 24 | VL | EIVLTQSPGTLSLSPGERATLSCRTSQSIN |
| | | AA | AILLAWYQQKPGQAPRLLIYGASSRATG |
| | | | IPDRFSGSGSGTDFTLSISRLEPEDFAVYY |
| | | | CQQYGSSPRTFGQGTKVEIK |
| | SEQ ID NO: 27 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTACTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATACAACTGGTTCGACCCCTGG |
| | | | GGCCAGGGAACCCTGGTCACCGTCTCC |
| | | | TCA |
| | SEQ ID NO: 25 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTAACGCCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| BGA-5488 | SEQ ID NO: 11 | HCDR1 | TSNYWN |
| | | (Kabat) | |
| | SEQ ID NO: 2 | HCDR2 | EIYPTGNTNYSPSLKS |
| | | (Kabat) | |
| | SEQ ID NO: 28 | HCDR3 | GYNYFDP |
| | | (Kabat) | |
| | SEQ ID NO: 23 | LCDR1 | RTSQSINAILLA |
| | | (Kabat) | |
| | SEQ ID NO: 5 | LCDR2 | GASSRAT |
| | | (Kabat) | |
| | SEQ ID NO: 6 | LCDR3 | QQYGSSPRT |
| | | (Kabat) | |
| | SEQ ID NO: 29 | VH | QVQLQESGPGLVKPSGTLSLTCAVSGGS |
| | | AA | ISTSNYWNWVRQSPGKGLEWIGEIYPTG |
| | | | NTNYSPSLKSRVTISIDKSKNQFSLKLNS |
| | | | VTAADTAVYYCARGYNYFDPWGQGTL |
| | | | VTVSS |
| | SEQ ID NO: 24 | VL | EIVLTQSPGTLSLSPGERATLSCRTSQSIN |
| | | AA | AILLAWYQQKPGQAPRLLIYGASSRATG |
| | | | IPDRFSGSGSGTDFTLSISRLEPEDFAVYY |
| | | | CQQYGSSPRTFGQGTKVEIK |
| | SEQ ID NO: 30 | VH | CAGGTGCAGCTGCAGGAGTCGGGCCC |
| | | DNA | AGGACTGGTGAAGCCTTCGGGGACCC |
| | | | TGTCCCTCACCTGCGCTGTCTCTGGTG |
| | | | GCTCCATCAGTACTAGTAACTACTGGA |
| | | | ATTGGGTCCGCCAGTCCCCAGGGAAG |
| | | | GGGCTGGAGTGGATTGGAGAAATCTA |
| | | | TCCTACTGGGAACACCAACTACAGCCC |
| | | | GTCCCTCAAGAGTCGAGTCACCATATC |
| | | | AATAGACAAGTCCAAGAACCAGTTCT |
| | | | CCCTGAAACTTAATTCTGTGACCGCCG |
| | | | CGGACACGGCCGTGTATTATTGTGCCA |
| | | | GAGGATACAACTACTTCGACCCCTGG |
| | | | GGCCAGGGAACCCTGGTCACCGTCTCC |
| | | | TCA |
| | SEQ ID NO: 25 | VL | GAAATTGTGTTGACGCAGTCTCCAGGC |
| | | DNA | ACCCTGTCTTTGTCTCCAGGGGAAAGA |
| | | | GCCACCCTCTCCTGCAGGACCAGTCAG |
| | | | AGTATTAACGCCATCTTGTTAGCCTGG |
| | | | TACCAGCAGAAACCTGGCCAGGCTCC |
| | | | CAGGCTCCTCATCTATGGTGCATCCAG |
| | | | CAGGGCCACTGGCATCCCCGACAGGT |
| | | | TCAGTGGCAGTGGGTCTGGGACAGAC |
| | | | TTCACTCTCAGCATCAGCAGACTGGAG |
| | | | CCTGAAGATTTTGCAGTGTATTACTGT |
| | | | CAGCAGTATGGCAGTTCACCTCGGAC |
| | | | GTTCGGCCAAGGGACCAAGGTGGAAA |
| | | | TCAAA |
| IgG1mf | SEQ ID NO: 31 | AA | ASTKGPSVFPLAPSSKSTSGGTAALGCL |
| | | | VKDYFPEPVTVSWNSGALTSGVHTFPA |
| | | | VLQSSGLYSLSSVVTVPSSSLGTQTYICN |
| | | | VNHKPSNTKVDKKVEPKSCDKTHTCPP |
| | | | CPAPPAAGPSVFLFPPKPKDTLMISRTPE |
| | | | VTCVVVDVSHEDPEVKFNWYVDGVEV |
| | | | HNAKTKPREEQYNSTYRVVSVLTVLHQ |
| | | | DWLNGKEYKCKVSNKALAAPIEKTISK |
| | | | AKGQPREPQVYTLPPSRDELTKNQVSLT |
| | | | CLVKGFYPSDIAVEWESNGQPENNYKTT |
| | | | PPVLDSDGSFFLYSKLTVDKSRWQQGN |
| | | | VFSCSVMHEALHNHYTQKSLSLSPGK |
| Heavy | SEQ ID NO: 33 | AA | ASTKGPSVFPLAPSSKSTSGGTAALGCL |
| chain | | | VKDYFPEPVTVSWNSGALTSGVHTFPA |
| constant | | | VLQSSGLYSLSSVVTVPSSSLGTQTYICN |
| region | | | VNHKPSNTKVDKKVEPKSCDKTHTCPP |
| from IgG1 | | | CPAPELLGGPSVFLFPPKPKDTLMISRTP |
| | | | EVTCVVVDVSHEDPEVKFNWYVDGVE |
| | | | VHNAKTKPREEQYNSTYRVVSVLTVLH |
| | | | QDWLNGKEYKCKVSNKALPAPIEKTISK |
| | | | AKGQPREPQVYTLPPSRDELTKNQVSLT |
| | | | CLVKGFYPSDIAVEWESNGQPENNYKTT |
| | | | PPVLDSDGSFFLYSKLTVDKSRWQQGN |
| | | | VFSCSVMHEALHNHYTQKSLSLSPGK |
| Light chain | SEQ ID NO: 34 | AA | RTVAAPSVFIFPPSDEQLKSGTASVVCLL |
| constant | | | NNFYPREAKVQWKVDNALQSGNSQES |
| region of | | | VTEQDSKDSTYSLSSTLTLSKADYEKHK |
| kappa | | | VYACEVTHQGLSSPVTKSFNRGEC |
| chain | | | |
| VH of | SEQ ID NO: 78 | AA | QVQLQESGPGLVKPSETLSLTCTVSGFSL |
| Tislelizumab | | | TSYGVHWIRQPPGKGLEWIGVIYADGST |
| | | | NYNPSLKSRVTISKDTSKNQVSLKLSSV |
| | | | TAADTAVYYCARAYGNYWYIDVWGQG |
| | | | TTVTVSS |
| VL of | SEQ ID NO: 79 | AA | DIVMTQSPDSLAVSLGERATINCKSSESV |
| Tislelizumab | | | SNDVAWYQQKPGQPPKLLINYAFHRFT |
| | | | GVPDRFSGSGYGTDFTLTISSLQAEDVA |
| | | | VYYCHQAYSSPYTFGQGTKLEIK |
| Full-length | SEQ ID NO: 80 | AA | MLRRRGSPGMGVHVGAALGALWFCLT |
| human | | | GALEVQVPEDPVVALVGTDATLCCSFSP |
| B7H3 | | | EPGFSLAQLNLIWQLTDTKQLVHSFAEG |
| | | | QDQGSAYANRTALFPDLLAQGNASLRL |
| | | | QRVRVADEGSFTCFVSIRDFGSAAVSLQ |
| | | | VAAPYSKPSMTLEPNKDLRPGDTVTITC |
| | | | SSYQGYPEAEVFWQDGQGVPLTGNVTT |
| | | | SQMANEQGLFDVHSILRVVLGANGTYS |
| | | | CLVRNPVLQQDAHSSVTITPQRSPTGAV |
| | | | EVQVPEDPVVALVGTDATLRCSFSPEPG |
| | | | FSLAQLNLIWQLTDTKQLVHSFTEGRDQ |
| | | | GSAYANRTALFPDLLAQGNASLRLQRV |
| | | | RVADEGSFTCFVSIRDFGSAAVSLQVAA |
| | | | PYSKPSMTLEPNKDLRPGDTVTITCSSYR |
| | | | GYPEAEVFWQDGQGVPLTGNVTTSQM |
| | | | ANEQGLFDVHSVLRVVLGANGTYSCLV |
| | | | RNPVLQQDAHGSVTITGQPMTFPPEAL |
| | | | WVTVGLSVCLIALLVALAFVCWRKIKQ |
| | | | SCEEENAGAEDQDGEGEGSKTALQPLK |
| | | | HSDSKEDDGQEIA |

### Example 6. Cellular Binding and Internalization of Humanized Ab

### Binding

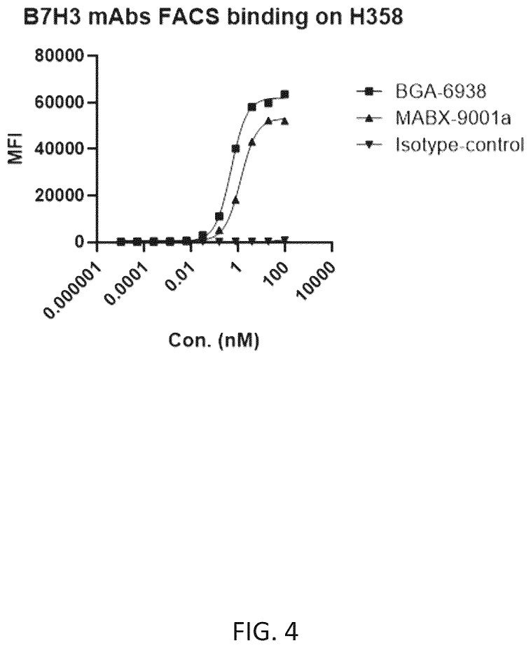

A binding analysis was performed to evaluate affinity for the antibody. BGA-6938 in wildtype IgG1/kappa format and MABX-9001a (a humanized anti-B7H3 IgG1 monoclonal antibody used for DS-7300a, Yamato et al., Mol Cancer Ther 2022 1; 21 (4): 635-646) and a human IgG antibody were included for the evaluation. The H358 cells were detached from flasks using Trypsin-EDTA (Cat. No. 25200056, ThermoFisher) and were washed with binding buffer (PBS with 2% FBS, pH 7.2). The washed cells were added at an approximate density of 100,000 cells in 0.2 mL of binding buffer to the 96-well plates containing the antibody. The final concentration of the primary antibody in the competition reaction with cells varied, starting at 500 nM and then decreasing by 1:5-fold dilution for eleven concentrations. The cell and antibody mixture were incubated for 1 hour on ice. After the 1-hour incubation, cells were washed twice with binding buffer to separate the free antibody. The washed cell was further labeled with florescence labeled anti-human Fc (Cat. No: 109-605-190. Jackson ImmunoResearch) and incubated for 1 hour on ice. After the 1-hour incubation, cells were washed twice with binding buffer to separate the free antibody. Florescence signal was detected using the LSRFortessa (BD). The binding curve for BGA-6938 and MABX-9001a and a human IgG antibody on H358 was shown in FIG. 4. Based on the binding results, BGA-6938 showed higher binding affinity to B7H3 compared with MABX-9001a.

### Internalization

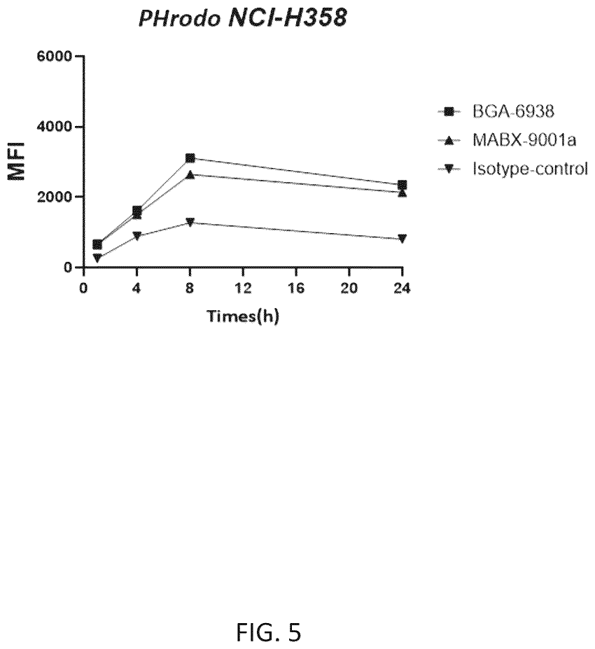

One desirable attribute of an antibody for conjugation to an ADC is the ability to internalize into the cell. A pHrodo™ assay (Cat No.: Z25612, ThermoFisher) according to manufacturer's protocol was performed to determine the internalization activity of BGA-6938 and MABX-9001a. The primary antibody was labeled with Zenon™ pHrodo™ iFL IgG Labeling Reagent (Cat. No.: Z25612, ThermoFisher) and RPMI medium. 100,000 H358 cells were suspended and incubated with antibody-pHrodo in 0.2 mL at 37° C. After the 1-hour, 4-hours, 8 hours and 24 hours incubation, cells were washed twice with binding buffer (PBS with 2% FBS, pH 7.2) to separate the free antibody. Florescence signal was detected the LSRFortessa™ (BD) with PE channel. The florescence signal intensity signifies the internalization activity. The curve for BGA-6938 and MABX-9001a and a human IgG antibody on H358 was shown in FIG. 5. Based on the internalization results, BGA-6938 showed increased internalization activity compared with MABX-9001a.

### Example 7. Cellular Killing Activity

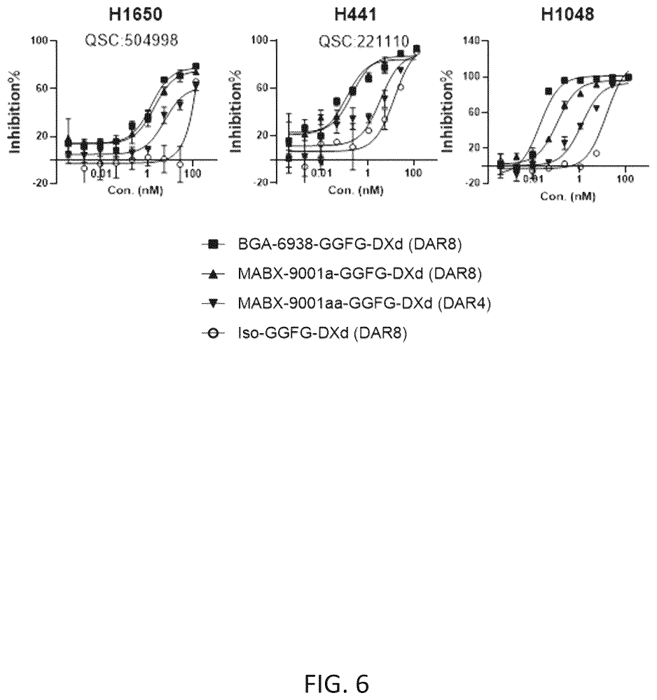

To assess the ADC cellular killing activity of BGA-6938 and MAbX-9001a, target cells H1650, H441 and H1048 (all lung cancer cell lines) were plated at 1,000 cells per well in 100 μl of RPM1-1640 plus 10% FBS medium in 96-well 3D cell culture plates (Cat. No.: 6055330, Perkin Elmer). Twenty-four hours later, an additional 50 μl of culture medium with serial dilutions of BGA-6938-GGFG-DXd (DAR8) and MABX-9001a-GGFG-DXd (DAR8 or DAR4) concentrations were added to triplicate wells. See FIG. 2A in Yamato et al., Mol Cancer Ther 2022 1; 21 (4): 635-646 for structure of MABX-9001a-GGFG-DXd. Six days later, cell survival was determined using CellTiter-Glo Luminescent Cell Viability Reagent (G7572; Promega Corporation) and with an Tecan Spark. The killing curve was analyzed with GraphPad Prism 9 and is shown in FIG. 6. BGA-6938-GGFG-DXd (DAR8) showed comparable killing activity in H1650 and H441 and increased killing activity in H1048 compared with MABX-9001a-GGFG-DXd (DAR8).

### Example 8. Binding of B7H3 scFv Protein on H358 Tumor Cell Line

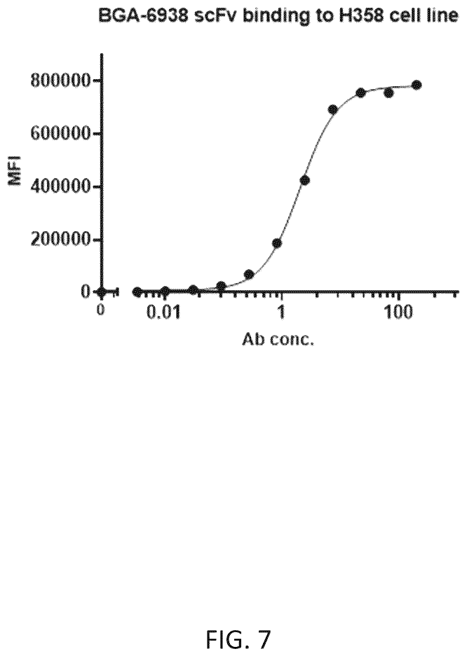

To evaluate the binding ability of scFv of BGA-6938 on the surface of living H358 tumor cells, scFv proteins (see Table 8) were purified via affinity chromatography followed by size-exclusion chromatography. For the assay, 2×10⁵ H358 cells were seeded into 96-well plate. The dose response curve was generated by adding serially diluted scFv proteins (3.38 PM to 200 nM) to cells and the bound scFv were detected using His-tag antibody iFluor 488 (Cat. No. A01800). MFI of each cell population were measured using Satorius iQue3™ system. EC50 and MFImax of each scFv were determined using four parameter logistic model. The results are shown in Table 9 and FIG. 7. ScFv of BGA-6938 showed an EC50 of 2.047 nM to B7H3 antigen on H358 tumor cells.

TABLE 8
sequence of B7H3 scFv protein

| | | |
|---|---|---|
| scFv | SEQ ID | EIVLTQSPGTLSLSPGERATLSCRTSQ |
| of BGA- | NO: 32 | SINAILLAWYQQKPGQAPRLLIYGASS |
| 6938 | | RATGIPDRFSGSGSGTDFTLSISRLEP |
| VL-VH | | EDFAVYYCQQYGSSPRTFGQGTKVEIK |
| | | ggggsggggsggggsggggsQVQLQES |
| | | GPGLVKPSGTLSLTCAVSGGSISTSNY |
| | | WNWVRQSPGKGLEWIGEIYPTGNTNYS |
| | | PSLKSRVTISIDKSKNQFSLKLNSVTA |
| | | ADTAVYYCARGYNWFDPWGQGTLVTVS |
| | | SHHHHHH |

TABLE 9
Dose Dependent Binding of scFv Fragments on H358 Cell Line

| Antibody | EC50 (nM) | MFI |
|---|---|---|
| scFv of BGA-6938 | 2.047 | 782469 |

### Example 9. Epitope Mapping of Anti-B7H3 Antibodies

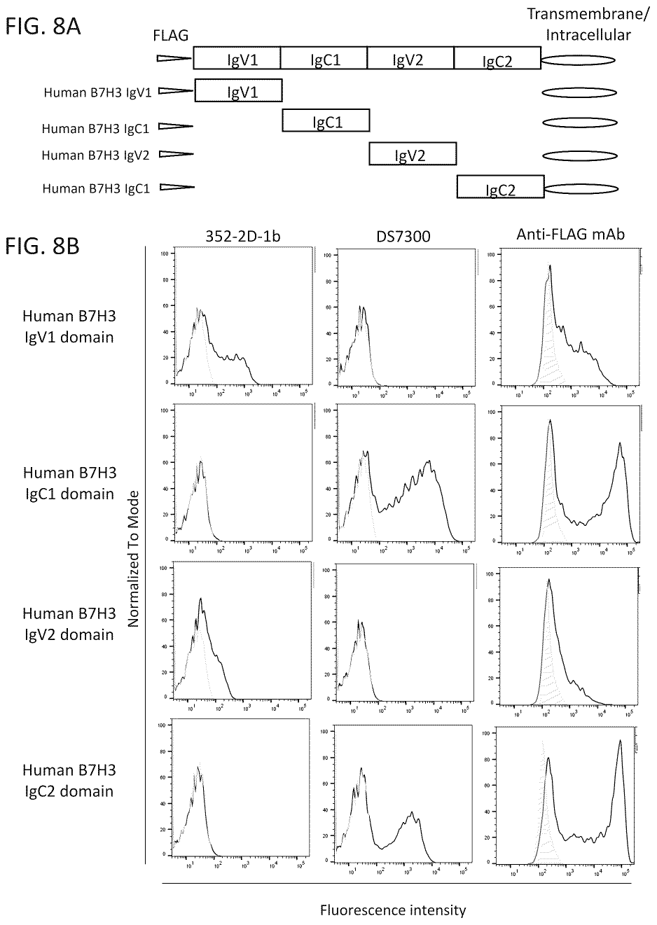

To study the binding epitope of anti-B7H3 mAb BGA-6938, domain truncated human B7-H3 was generated by fusing each Ig-like domain from extracellular regions of human B7H3 (SEQ ID NO: 80), namely IgV1 (amino acids 29-139 of SEQ ID NO: 80), IgC1 (amino acids 145-238 of SEQ ID NO: 80), IgV2 (amino acids 243-357 of SEQ ID NO: 80), and IgC2 (amino acids 363-456 of SEQ ID NO: 80) with its transmembrane and intracellular domain. The truncated version of B7H3 were fused with an N-terminal FLAG tag as well. The resulting truncated human B7H3 constructs shown in FIG. 8A contain an N-terminal FLAG tag followed by an Ig-like domain and B7H3 C-terminal domain including transmembrane and intracellular domains. The DNA encoding the truncated versions of B7H3 were cloned into pcDNA 3.4 vectors.

The plasmids containing these truncated B7H3 constructs were used to transfect ExpiCHO™ cells for transient protein expression, after which cells were incubated with 100 nM purified BGA-6938 as well as a reference antibody DS-7300 from Daiichi (US 2022/0064312 A1). The binding of BGA-6938 and DS-7300 with different B7H3 truncated forms was evaluated by detection with Alexa Fluor 647 Rabbit Anti-Human IgG (Cat.: 309-605-008 Jackson ImmunoResearch). The expression of each construct was validated by the detection of FLAG tag on the N-terminus after incubating transfected cells with anti-FLAG mAb (Cat.: A01809, Genscript). The data in FIG. 8B shows that BGA-6938 specifically binds to the B7H3 V1 and V2 domains and shows no binding to the B7H3 C1 and C2 domains. In comparison, reference antibody DS-7300 binds to the C1 and C2 domain of B7H3 and has no binding to the V1 and V2 domains (FIG. 8B). Accordingly, BGA-6938 and DS-7300 have non-overlapping epitopes.

1. An antibody, or antigen-binding fragment thereof, which is capable of specifically binding to human 4Ig-B7H3, comprising:
(i) a heavy chain variable region (VH) that comprises (a) a HCDR1 (Heavy Chain Complementarity Determining Region 1) of SEQ ID NO: 11, (b) a HCDR2 of SEQ ID NO: 2, and (c) a HCDR3 of SEQ ID NO: 14, and a light chain variable region (VL) that comprises (d) a LCDR1 (Light Chain Complementarity Determining Region 1) of SEQ ID NO: 23, (e) a LCDR2 of SEQ ID NO: 5, and (f) a LCDR3 of SEQ ID NO: 6;
(ii) a heavy chain variable region that comprises (a) a HCDR1 of SEQ ID NO: 1, (b) a HCDR2 of SEQ ID NO: 2, and (c) a HCDR3 of SEQ ID NO: 3, and a light chain variable region that comprises (d) a LCDR1 of SEQ ID NO: 4, (e) a LCDR2 of SEQ ID NO: 5, and (f) a LCDR3 of SEQ ID NO: 6;
(iii) a heavy chain variable region that comprises (a) a HCDR1 of SEQ ID NO: 11, (b) a HCDR2 of SEQ ID NO: 2, and (c) a HCDR3 of SEQ ID NO: 3, and a light chain variable region that comprises (d) a LCDR1 of SEQ ID NO: 4, (e) a LCDR2 of SEQ ID NO: 5, and (f) a LCDR3 of SEQ ID NO: 6;
(iv) a heavy chain variable region that comprises (a) a HCDR1 of SEQ ID NO: 1, (b) a HCDR2 of SEQ ID NO: 2, and (c) a HCDR3 of SEQ ID NO: 14, and a light chain variable region that comprises (d) a LCDR1 of SEQ ID NO: 4, (e) a LCDR2 of SEQ ID NO: 5, and (f) a LCDR3 of SEQ ID NO: 6;
(v) a heavy chain variable region that comprises (a) a HCDR1 of SEQ ID NO: 1, (b) a HCDR2 of SEQ ID NO: 2, and (c) a HCDR3 of SEQ ID NO: 17, and a light chain variable region that comprises (d) a LCDR1 of SEQ ID NO: 4, (e) a LCDR2 of SEQ ID NO: 5, and (f) a LCDR3 of SEQ ID NO: 6;
(vi) a heavy chain variable region that comprises (a) a HCDR1 of SEQ ID NO: 1, (b) a HCDR2 of SEQ ID NO: 2, and (c) a HCDR3 of SEQ ID NO: 3, and a light chain variable region that comprises (d) a LCDR1 of SEQ ID NO: 20, (e) a LCDR2 of SEQ ID NO: 5, and (f) a LCDR3 of SEQ ID NO: 6;
(vii) a heavy chain variable region that comprises (a) a HCDR1 of SEQ ID NO: 1, (b) a HCDR2 of SEQ ID NO: 2, and (c) a HCDR3 of SEQ ID NO: 3, and a light chain variable region that comprises (d) a LCDR1 of SEQ ID NO: 23, (e) a LCDR2 of SEQ ID NO: 5, and (f) a LCDR3 of SEQ ID NO: 6; or
(viii) a heavy chain variable region that comprises (a) a HCDR1 of SEQ ID NO: 11, (b) a HCDR2 of SEQ ID NO: 2, and (c) a HCDR3 of SEQ ID NO: 28, and a light chain variable region that comprises (d) a LCDR1 of SEQ ID NO: 23, (e) a LCDR2 of SEQ ID NO: 5, and (f) a LCDR3 of SEQ ID NO: 6.

2. The antibody, or antigen-binding fragment thereof, of claim 1, wherein:
(i) the heavy chain variable region comprises an amino acid sequence at least 90, 91, 92, 93, 94, 95, 96, 97, 98 or 99% identical to SEQ ID NO: 26, and the light chain variable region comprises an amino acid sequence at least 90, 91, 92, 93, 94, 95, 96, 97, 98 or 99% identical to SEQ ID NO: 24;
(ii) the heavy chain variable region comprises an amino acid sequence at least 90, 91, 92, 93, 94, 95, 96, 97, 98 or 99% identical to SEQ ID NO: 7, and the light chain variable region comprises an amino acid sequence at least 90, 91, 92, 93, 94, 95, 96, 97, 98 or 99% identical to SEQ ID NO: 8;
(iii) the heavy chain variable region comprises an amino acid sequence at least 90, 91, 92, 93, 94, 95, 96, 97, 98 or 99% identical to SEQ ID NO: 12, and the light chain variable region comprises an amino acid sequence at least 90, 91, 92, 93, 94, 95, 96, 97, 98 or 99% identical to SEQ ID NO: 8;
(iv) the heavy chain variable region comprises an amino acid sequence at least 90, 91, 92, 93, 94, 95, 96, 97, 98 or 99% identical to SEQ ID NO: 15, and the light chain variable region comprises an amino acid sequence at least 90, 91, 92, 93, 94, 95, 96, 97, 98 or 99% identical to SEQ ID NO: 8;
(v) the heavy chain variable region comprises an amino acid sequence at least 90, 91, 92, 93, 94, 95, 96, 97, 98 or 99% identical to SEQ ID NO: 18, and the light chain variable region comprises an amino acid sequence at least 90, 91, 92, 93, 94, 95, 96, 97, 98 or 99% identical to SEQ ID NO: 8;
(vi) the heavy chain variable region comprises an amino acid sequence at least 90, 91, 92, 93, 94, 95, 96, 97, 98 or 99% identical to SEQ ID NO: 7, and the light chain variable region comprises an amino acid sequence at least 90, 91, 92, 93, 94, 95, 96, 97, 98 or 99% identical to SEQ ID NO: 21;
(vii) the heavy chain variable region comprises an amino acid sequence at least 90, 91, 92, 93, 94, 95, 96, 97, 98 or 99% identical to SEQ ID NO: 7, and the light chain variable region comprises an amino acid sequence at least 90, 91, 92, 93, 94, 95, 96, 97, 98 or 99% identical to SEQ ID NO: 24; or
(viii) the heavy chain variable region comprises an amino acid sequence at least 90, 91, 92, 93, 94, 95, 96, 97, 98 or 99% identical to SEQ ID NO: 29, and the light chain variable region comprises an amino acid sequence at least 90, 91, 92, 93, 94, 95, 96, 97, 98 or 99% identical to SEQ ID NO: 24.

3. The antibody or antigen-binding fragment thereof of claim 2, wherein one, two, three, four, five, six, seven, eight, nine, or ten amino acids within SEQ ID NOs: 26 and 24, SEQ ID NOs: 7 and 8, SEQ ID NOs: 12 and 8, SEQ ID NOs: 15 and 8, SEQ ID NOs: 18 and 8, SEQ ID NOs: 7 and 21, SEQ ID Nos: 7 and 24, SEQ ID Nos: 29 and 24 have been inserted, deleted or substituted.

4. The antibody, or antigen-binding fragment thereof, of claim 1, wherein:
(i) the heavy chain variable region comprises SEQ ID NO: 26, and the light chain variable region comprises SEQ ID NO: 24;
(ii) the heavy chain variable region comprises SEQ ID NO: 7, and the light chain variable region comprises SEQ ID NO: 8;
(iii) the heavy chain variable region comprises SEQ ID NO: 12, and the light chain variable region comprises SEQ ID NO: 8;
(iv) the heavy chain variable region comprises SEQ ID NO: 15, and the light chain variable region comprises SEQ ID NO: 8;
(v) the heavy chain variable region comprises SEQ ID NO: 18, and the light chain variable region comprises SEQ ID NO: 8;
(vi) the heavy chain variable region comprises SEQ ID NO: 7, and the light chain variable region comprises SEQ ID NO: 21;
(vii) the heavy chain variable region comprises SEQ ID NO: 7, and the light chain variable region comprises SEQ ID NO: 24; or
(viii) the heavy chain variable region comprises SEQ ID NO: 29, and the light chain variable region comprises SEQ ID NO: 24.

5. The antibody, or antigen-binding fragment thereof, of claim 1, which is a monoclonal antibody, a human engineered antibody, a single chain antibody (scFv), a Fab fragment, a Fab′ fragment, or a F(ab′)₂ fragment.

6. The antibody, or antigen-binding fragment thereof, of claim 5, wherein the antibody, or antigen-binding fragment thereof, comprises a scFv comprising a VH having the amino acid of SEQ ID NO: 26, and a VL having an amino acid of SEQ ID NO: 24, optionally the VH and VL are connected via an amino acid linker, and optionally the amino acid linker is any sequence of SEQ ID NO: 35 to SEQ ID NO: 77.

7. (canceled)

8. The antibody, or antigen-binding fragment thereof, of claim 6, wherein the antibody or antigen-binding fragment thereof comprises a scFv having the amino acid sequence of SEQ ID NO: 32.

9. A multispecific antibody, or antigen-binding fragment thereof, comprising:
at least a first antigen binding domain that is capable of specifically binding to human 4Ig-B7H3, wherein the first antigen binding domain are the antibody, or antigen-binding fragment thereof, of claim 1; and
at least a second antigen binding domain that is capable of specifically binding to a second human tumor-associated antigen (TAA).

10. (canceled)

11. The multispecific antibody, or antigen-binding fragment thereof, of claim 9, further comprising an amino acid linker, wherein the amino acid linker is any sequence of SEQ ID NO: 35 to SEQ ID NO: 77.

12. The antibody, or antigen-binding fragment thereof, of claim 1, wherein the antibody, or antigen-binding fragment thereof, comprises a heavy chain constant region of the subclass of IgG1, IgG2, IgG3, or IgG4, and/or a light chain constant region of the type of kappa or lambda.

13. (canceled)

14. The antibody, or antigen-binding fragment thereof, of claim 1, wherein the antibody, or antigen-binding fragment thereof, has antibody dependent cellular cytotoxicity (ADCC) or complement dependent cytotoxicity (CDC).

15. The antibody, or antigen-binding fragment thereof, of claim 1, wherein the antibody, or antigen-binding fragment thereof, has reduced glycosylation or no glycosylation or is hypofucosylated.

16. The antibody, or antigen-binding fragment thereof, of claim 1, wherein the antibody, or antigen-binding fragment thereof, comprises increased bisecting GlcNac structures.

17. The antibody, or antigen-binding fragment thereof, of claim 1, wherein the antibody, or antigen-binding fragment thereof, is conjugated to a cytotoxin via a cytotoxin linker.

18-19. (canceled)

20. A pharmaceutical composition comprising the antibody, or antigen-binding fragment thereof, of claim 1, and a pharmaceutically acceptable carrier.

21. A method of treating a cancer comprising administering to a patient in need thereof an effective amount of the antibody, or antigen-binding fragment thereof, of claim 1.

22. (canceled)

23. The method of claim 21, wherein the cancer is colorectal carcinoma, prostate cancer, pancreatic cancer, breast cancer, ovarian cancer, kidney cancer, lung cancer, or esophageal carcinoma.

24. (canceled)

25. The method of claim 23, wherein the lung cancer is non-small cell lung cancer (NSCLC) or small cell lung cancer (SCLC), and wherein the esophageal carcinoma is esophageal squamous cell carcinoma.

26. (canceled)

27. The method of claim 21, wherein the antibody, or antigen-binding fragment thereof, is administered in combination with another therapeutic agent, and optionally wherein the therapeutic agent is paclitaxel or a paclitaxel agent, docetaxel, carboplatin, topotecan, cisplatin, irinotecan, doxorubicin, lenalidomide, 5-azacytidine, an immune checkpoint inhibitor, or an anti-PD-1 antibody, optionally wherein the anti-PD-1 antibody is tislelizumab.

28-31. (canceled)

32. An isolated nucleic acid that encodes the antibody, or antigen-binding fragment thereof, of claim 1.

33-36. (canceled)


SEQUENCE LISTING (80 sequences)

SEQ ID NO: 1 - protein, 6 aa, synthetic construct
  source 1..6: note = HCDR1 (Kabat)
TSNWWN

SEQ ID NO: 2 - protein, 16 aa, synthetic construct
  source 1..16: note = HCDR2 (Kabat)
EIYPTGNTNYSPSLKS

SEQ ID NO: 3 - protein, 7 aa, synthetic construct
  source 1..7: note = HCDR3 (Kabat)
GWNWFDP

SEQ ID NO: 4 - protein, 12 aa, synthetic construct
  source 1..12: note = LCDR1 (Kabat)
RTSQSINGILLA

SEQ ID NO: 5 - protein, 7 aa, synthetic construct
  source 1..7: note = LCDR2 (Kabat)
GASSRAT

SEQ ID NO: 6 - protein, 9 aa, synthetic construct
  source 1..9: note = LCDR3 (Kabat)
QQYGSSPRT

SEQ ID NO: 7 - protein, 116 aa, synthetic construct
  source 1..116: note = VH AA
QVQLQESGPGLVKPSGTLSLTCAVSGGSISTSNWWNWVRQSPGKGLEWIGEIYPTGNTNYSPSLKSRVTISIDKSKNQFSLKLNSVTAADTAVYYCARGWNWFDPWGQGTLVTVSS

SEQ ID NO: 8 - protein, 108 aa, synthetic construct
  source 1..108: note = VL AA
EIVLTQSPGTLSLSPGERATLSCRTSQSINGILLAWYQQKPGQAPRLLIYGASSRATGIPDRFSGSGSGTDFTLSISRLEPEDFAVYYCQQYGSSPRTFGQGTKVEIK

SEQ ID NO: 9 - DNA, 348 bp, synthetic construct
  source 1..348: mol_type = other DNA; note = VH DNA
caggtgcagctgcaggagtcgggcccaggactggtgaagccttcggggaccctgtccctcacctgcgctgtctctggtggctccatcagtactagtaactggtggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatggaactggttcgacccctggggccagggaaccctggtcaccgtctcctca

SEQ ID NO: 10 - DNA, 324 bp, synthetic construct
  source 1..324: mol_type = other DNA; note = VL DNA
gaaattgtgttgacgcagtctccaggcaccctgtctttgtctccaggggaaagagccaccctctcctgcaggaccagtcagagtattaacggcatcttgttagcctggtaccagcagaaacctggccaggctcccaggctcctcatctatggtgcatccagcagggccactggcatccccgacaggttcagtggcagtgggtctgggacagacttcactctcagcatcagcagactggagcctgaagattttgcagtgtattactgtcagcagtatggcagttcacctcggacgttcggccaagggaccaaggtggaaatcaaa

SEQ ID NO: 11 - protein, 6 aa, synthetic construct
  source 1..6: note = HCDR1 (Kabat)
TSNYWN

SEQ ID NO: 12 - protein, 116 aa, synthetic construct
  source 1..116: note = VH AA
QVQLQESGPGLVKPSGTLSLTCAVSGGSISTSNYWNWVRQSPGKGLEWIGEIYPTGNTNYSPSLKSRVTISIDKSKNQFSLKLNSVTAADTAVYYCARGWNWFDPWGQGTLVTVSS

SEQ ID NO: 13 - DNA, 348 bp, synthetic construct
  source 1..348: mol_type = other DNA; note = VH DNA
caggtgcagctgcaggagtcgggcccaggactggtgaagccttcggggaccctgtccctcacctgcgctgtctctggtggctccatcagtactagtaactactggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatggaactggttcgacccctggggccagggaaccctggtcaccgtctcctca

SEQ ID NO: 14 - protein, 7 aa, synthetic construct
  source 1..7: note = HCDR3 (Kabat)
GYNWFDP

SEQ ID NO: 15 - protein, 116 aa, synthetic construct
  source 1..116: note = VH AA
QVQLQESGPGLVKPSGTLSLTCAVSGGSISTSNWWNWVRQSPGKGLEWIGEIYPTGNTNYSPSLKSRVTISIDKSKNQFSLKLNSVTAADTAVYYCARGYNWFDPWGQGTLVTVSS

SEQ ID NO: 16 - DNA, 348 bp, synthetic construct
  source 1..348: mol_type = other DNA; note = VH DNA
caggtgcagctgcaggagtcgggcccaggactggtgaagccttcggggaccctgtccctcacctgcgctgtctctggtggctccatcagtactagtaactggtggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatacaactggttcgacccctggggccagggaaccctggtcaccgtctcctca

SEQ ID NO: 17 - protein, 7 aa, synthetic construct
  source 1..7: note = HCDR3 (Kabat)
GWNYFDP

SEQ ID NO: 18 - protein, 116 aa, synthetic construct
  source 1..116: note = VH AA
QVQLQESGPGLVKPSGTLSLTCAVSGGSISTSNWWNWVRQSPGKGLEWIGEIYPTGNTNYSPSLKSRVTISIDKSKNQFSLKLNSVTAADTAVYYCARGWNYFDPWGQGTLVTVSS

SEQ ID NO: 19 - DNA, 348 bp, synthetic construct
  source 1..348: mol_type = other DNA; note = VH DNA
caggtgcagctgcaggagtcgggcccaggactggtgaagccttcggggaccctgtccctcacctgcgctgtctctggtggctccatcagtactagtaactggtggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatggaactacttcgacccctggggccagggaaccctggtcaccgtctcctca

SEQ ID NO: 20 - protein, 12 aa, synthetic construct
  source 1..12: note = LCDR1 (Kabat)
RTSQSIQGILLA

SEQ ID NO: 21 - protein, 108 aa, synthetic construct
  source 1..108: note = VL AA
EIVLTQSPGTLSLSPGERATLSCRTSQSIQGILLAWYQQKPGQAPRLLIYGASSRATGIPDRFSGSGSGTDFTLSISRLEPEDFAVYYCQQYGSSPRTFGQGTKVEIK

SEQ ID NO: 22 - DNA, 324 bp, synthetic construct
  source 1..324: mol_type = other DNA; note = VL DNA
gaaattgtgttgacgcagtctccaggcaccctgtctttgtctccaggggaaagagccaccctctcctgcaggaccagtcagagtattcagggcatcttgttagcctggtaccagcagaaacctggccaggctcccaggctcctcatctatggtgcatccagcagggccactggcatccccgacaggttcagtggcagtgggtctgggacagacttcactctcagcatcagcagactggagcctgaagattttgcagtgtattactgtcagcagtatggcagttcacctcggacgttcggccaagggaccaaggtggaaatcaaa

SEQ ID NO: 23 - protein, 12 aa, synthetic construct
  source 1..12: note = LCDR1 (Kabat)
RTSQSINAILLA

SEQ ID NO: 24 - protein, 108 aa, synthetic construct
  source 1..108: note = VL AA
EIVLTQSPGTLSLSPGERATLSCRTSQSINAILLAWYQQKPGQAPRLLIYGASSRATGIPDRFSGSGSGTDFTLSISRLEPEDFAVYYCQQYGSSPRTFGQGTKVEIK

SEQ ID NO: 25 - DNA, 324 bp, synthetic construct
  source 1..324: mol_type = other DNA; note = VL DNA
gaaattgtgttgacgcagtctccaggcaccctgtctttgtctccaggggaaagagccaccctctcctgcaggaccagtcagagtattaacgccatcttgttagcctggtaccagcagaaacctggccaggctcccaggctcctcatctatggtgcatccagcagggccactggcatccccgacaggttcagtggcagtgggtctgggacagacttcactctcagcatcagcagactggagcctgaagattttgcagtgtattactgtcagcagtatggcagttcacctcggacgttcggccaagggaccaaggtggaaatcaaa

SEQ ID NO: 26 - protein, 116 aa, synthetic construct
  source 1..116: note = VH AA
QVQLQESGPGLVKPSGTLSLTCAVSGGSISTSNYWNWVRQSPGKGLEWIGEIYPTGNTNYSPSLKSRVTISIDKSKNQFSLKLNSVTAADTAVYYCARGYNWFDPWGQGTLVTVSS

SEQ ID NO: 27 - DNA, 348 bp, synthetic construct
  source 1..348: mol_type = other DNA; note = VH DNA
caggtgcagctgcaggagtcgggcccaggactggtgaagccttcggggaccctgtccctcacctgcgctgtctctggtggctccatcagtactagtaactactggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatacaactggttcgacccctggggccagggaaccctggtcaccgtctcctca

SEQ ID NO: 28 - protein, 7 aa, synthetic construct
  source 1..7: note = HCDR3 (Kabat)
GYNYFDP

SEQ ID NO: 29 - protein, 116 aa, synthetic construct
  source 1..116: note = VH AA
QVQLQESGPGLVKPSGTLSLTCAVSGGSISTSNYWNWVRQSPGKGLEWIGEIYPTGNTNYSPSLKSRVTISIDKSKNQFSLKLNSVTAADTAVYYCARGYNYFDPWGQGTLVTVSS

SEQ ID NO: 30 - DNA, 348 bp, synthetic construct
  source 1..348: mol_type = other DNA; note = VH DNA
caggtgcagctgcaggagtcgggcccaggactggtgaagccttcggggaccctgtccctcacctgcgctgtctctggtggctccatcagtactagtaactactggaattgggtccgccagtccccagggaaggggctggagtggattggagaaatctatcctactgggaacaccaactacagcccgtccctcaagagtcgagtcaccatatcaatagacaagtccaagaaccagttctccctgaaacttaattctgtgaccgccgcggacacggccgtgtattattgtgccagaggatacaactacttcgacccctggggccagggaaccctggtcaccgtctcctca

SEQ ID NO: 31 - protein, 329 aa, synthetic construct
  source 1..329: note = IgG1mf
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 250 aa, synthetic construct
  source 1..250: note = scFv of BGA-6938 VL-VH
EIVLTQSPGTLSLSPGERATLSCRTSQSINAILLAWYQQKPGQAPRLLIYGASSRATGIPDRFSGSGSGTDFTLSISRLEPEDFAVYYCQQYGSSPRTFGQGTKVEIKGGGGSGGGGSGGGGSGGGGSQVQLQESGPGLVKPSGTLSLTCAVSGGSISTSNYWNWVRQSPGKGLEWIGEIYPTGNTNYSPSLKSRVTISIDKSKNQFSLKLNSVTAADTAVYYCARGYNWFDPWGQGTLVTVSSHHHHHH

SEQ ID NO: 33 - protein, 330 aa, synthetic construct [sequence omitted]

SEQ ID NO: 34 - protein, 107 aa, synthetic construct [sequence omitted]

SEQ ID NO: 35-39 - skipped (5 entries)

SEQ ID NO: 40 - protein, 4 aa, synthetic construct
  source 1..4: note = Linker
GGGS

SEQ ID NO: 41 - protein, 4 aa, synthetic construct
  source 1..4: note = Linker
SGGG

SEQ ID NO: 42 - protein, 5 aa, synthetic construct
  source 1..5: note = Linker
GGGGS

SEQ ID NO: 43 - protein, 7 aa, synthetic construct
  source 1..7: note = Linker
GGGGSGS

SEQ ID NO: 44 - protein, 7 aa, synthetic construct
  source 1..7: note = Linker
GGGGSGS

SEQ ID NO: 45 - protein, 8 aa, synthetic construct
  source 1..8: note = Linker
GGGGSGGS

SEQ ID NO: 46 - protein, 10 aa, synthetic construct
  source 1..10: note = Linker
GGGGSGGGGS

SEQ ID NO: 47 - protein, 15 aa, synthetic construct
  source 1..15: note = Linker
GGGGSGGGGSGGGGS

SEQ ID NO: 48 - protein, 16 aa, synthetic construct
  source 1..16: note = Linker
AKTTPKLEEGEFSEAR

SEQ ID NO: 49 - protein, 17 aa, synthetic construct
  source 1..17: note = Linker
AKTTPKLEEGEFSEARV

SEQ ID NO: 50 - protein, 9 aa, synthetic construct
  source 1..9: note = Linker
AKTTPKLGG

SEQ ID NO: 51 - protein, 10 aa, synthetic construct
  source 1..10: note = Linker
SAKTTPKLGG

SEQ ID NO: 52 - protein, 17 aa, synthetic construct
  source 1..17: note = Linker
AKTTPKLEEGEFSEARV

SEQ ID NO: 53 - protein, 6 aa, synthetic construct
  source 1..6: note = Linker
SAKTTP

SEQ ID NO: 54 - protein, 10 aa, synthetic construct
  source 1..10: note = Linker
SAKTTPKLGG

SEQ ID NO: 55 - protein, 6 aa, synthetic construct
  source 1..6: note = Linker
RADAAP

SEQ ID NO: 56 - protein, 9 aa, synthetic construct
  source 1..9: note = Linker
RADAAPTVS

SEQ ID NO: 57 - protein, 12 aa, synthetic construct
  source 1..12: note = Linker
RADAAAAGGPGS

SEQ ID NO: 58 - protein, 27 aa, synthetic construct
  source 1..27: note = Linker
RADAAAAGGGGSGGGGSGGGGSGGGGS

SEQ ID NO: 59 - protein, 6 aa, synthetic construct
  source 1..6: note = Linker
SAKTTP

SEQ ID NO: 60 - protein, 10 aa, synthetic construct
  source 1..10: note = Linker
SAKTTPKLGG

SEQ ID NO: 61 - protein, 18 aa, synthetic construct
  source 1..18: note = Linker
SAKTTPKLEEGEFSEARV

SEQ ID NO: 62 - protein, 5 aa, synthetic construct
  source 1..5: note = Linker
ADAAP

SEQ ID NO: 63 - protein, 12 aa, synthetic construct
  source 1..12: note = Linker
ADAAPTVSIFPP

SEQ ID NO: 64 - protein, 5 aa, synthetic construct
  source 1..5: note = Linker
TVAAP

SEQ ID NO: 65 - protein, 12 aa, synthetic construct
  source 1..12: note = Linker
TVAAPSVFIFPP

SEQ ID NO: 66 - protein, 6 aa, synthetic construct
  source 1..6: note = Linker
QPKAAP

SEQ ID NO: 67 - protein, 13 aa, synthetic construct
  source 1..13: note = Linker
QPKAAPSVTLFPP

SEQ ID NO: 68 - protein, 6 aa, synthetic construct
  source 1..6: note = Linker
AKTTPP

SEQ ID NO: 69 - protein, 13 aa, synthetic construct
  source 1..13: note = Linker
AKTTPPSVTPLAP

SEQ ID NO: 70 - protein, 6 aa, synthetic construct
  source 1..6: note = Linker
AKTTAP

SEQ ID NO: 71 - protein, 13 aa, synthetic construct
  source 1..13: note = Linker
AKTTAPSVYPLAP

SEQ ID NO: 72 - protein, 6 aa, synthetic construct
  source 1..6: note = Linker
ASTKGP

SEQ ID NO: 73 - protein, 13 aa, synthetic construct
  source 1..13: note = Linker
ASTKGPSVFPLAP

SEQ ID NO: 74 - protein, 15 aa, synthetic construct
  source 1..15: note = Linker
GENKVEYAPALMALS

SEQ ID NO: 75 - protein, 15 aa, synthetic construct
  source 1..15: note = Linker
GPAKELTPLKEAKVS

SEQ ID NO: 76 - protein, 15 aa, synthetic construct
  source 1..15: note = Linker
GHEAAAVMQVQYPAS

SEQ ID NO: 77 - protein, 20 aa, synthetic construct
  source 1..20: note = Linker
GGGGSGGGGSGGGGSGGGGS

SEQ ID NO: 78 - protein, 118 aa, synthetic construct
  source 1..118: note = VH of Tislelizumab
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 79 - protein, 107 aa, synthetic construct
  source 1..107: note = VL of Tislelizumab
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 80 - protein, 534 aa, synthetic construct
  source 1..534: note = Full-length human B7H3
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA